(19) United States
(12) Patent Application Publication
(10) Pub. No.: US 2025/0327823 A1
(43) Pub. Date: Oct. 23, 2025

(54) METHOD FOR THE IN VITRO DIAGNOSIS OF NEUROGENERATIVE METABOLIC DISEASES

(71) Applicant: OSPEDALE PEDIATRICO BAMBINO GESU', Roma (IT)

(72) Inventors: Carlo DIONISI VICI, Roma (IT); Anna SIDORINA, Roma (IT); Giulio CATESINI, Roma (IT); Cristiano RIZZO, Roma (IT)

(73) Assignee: OSPEDALE PEDIATRICO BAMBINO GESU', Roma, RM (IT)

(21) Appl. No.: 18/870,956

(22) PCT Filed: Jun. 1, 2022
(86) PCT No.: PCT/IT2022/000025

§ 371 Date: Dec. 2, 2024

(51) Int. Cl. G01N 33/92; G01N 33/94

(52) U.S. Cl. CPC G01N 33/92; G01N 33/948; G01N 2800/2835

(57) ABSTRACT

The present invention concerns a method for the in vitro diagnosis of neurodegenerative metabolic diseases able to simultaneously identify biomarkers characteristic of a large number neurodegenerative metabolic diseases.

The present invention concerns a method for the in vitro diagnosis of neurodegenerative metabolic diseases. In particular, the present invention concerns a method for the in vitro diagnosis of neurodegenerative metabolic diseases able to simultaneously identify biomarkers characteristic of a large number neurodegenerative metabolic diseases.

It is well known that inherited metabolic disorders (IMD) are genetic conditions that alter metabolic cellular pathways resulting in a large panel of clinical manifestation that affect multiple organ and systems. Those with a major involvement of Central Nervous System can be defined as “neurodegenerative metabolic diseases”.

IMD are individually rare but collectively numerous, and many diseases are, or are becoming, treatable. The therapeutical approaches for neurodegenerative metabolic diseases include enzyme replacement therapies, hematopoietic stem cell transplantation, specific drugs, and gene therapy [1,2].

A recently proposed nosology of IMD included 1450 disorders divided in 24 categories comprising 124 groups [3]. Among these categories, the great majority of metabolic neurodegenerative disorders refers to
complex molecules degradation;
lipid metabolism and transport;
peroxisomal fatty acid oxidation;
peroxisomal biogenesis disorders.

Since the clinical phenotypes are very complex and heterogeneous, this often represents a major issue causing delayed diagnosis.

The metabolic neurodegenerative disorders can be diagnosed through
selective screening driven by clinical signs:
1. identification of specific biomarkers in biological fluids;
2. measurement of the enzyme activity;
3. identification of genetic mutation by molecular analysis;
newborn screening (NBS):
1. measurement of the enzyme activity in DBS and/or
2. identification of specific biomarkers in dried blood spot (DBS).

Table 1 reported below illustrates a list of the most common metabolic neurodegenerative diseases, the individual enzymatic defect and the diagnostic biomarkers along with MIM number.

TABLE 1

| | | | | | |
|---|---|---|---|---|---|
| | | Phenotype | | OMIM | |
| Neurodegenerative | Enzyme/protein | MIM | | gene | |
| disease | deficiency | number | Gene | number | biomarkers |

Disorders of sphingolipid degradation

| | | | | | |
|---|---|---|---|---|---|
| Fabry | Alpha-galactosidase A | 301500 | GLA | 300644 | lysoGB3 |
| disease | | | | | |
| Gaucher | Lysosomal | 608013, | GBA | 606463 | lysoGlcSph |
| disease | glucosylceramidase | 230800, | | | |
| | | 230900, | | | |
| | | 231000, | | | |
| | | 231005 | | | |
| Krabbe | Beta- | 245200 | GALC | 606890 | lysoGalSph |
| disease | galactosylceramidase | | | | |
| Beta- | Beta-galactosidase | 230500, | GLB1 | 611458 | lysoGM1 |
| galactosidase | | 230600, | | | |
| deficiency, | | 230650, | | | |
| GM1 | | 253010 | | | |
| gangliosidosis | | | | | |
| phenotype | | | | | |
| GM2 | Beta-hexosaminidase | 272800 | HEXA | 606869 | lysoGM2 |
| gangliosidosis, | subunit alpha | | | | |
| B variant | | | | | |
| (Tay-Sachs | | | | | |
| disease) | | | | | |
| GM2 | Beta-hexosaminidase | 268800 | HEXB | 606873 | lysoGM2 |
| gangliosidosis, | subunit beta | | | | |
| O variant | | | | | |
| (Sandhoff | | | | | |
| disease) | | | | | |
| Acid | Sphingomyelin | 257200, | SMPD1 | 607608 | lysoSM, |
| sphingomyelinase | phosphodiesterase 1 | 607616 | | | lysoSM-509 |
| deficiency | | | | | |
| (ASMD) | | | | | |
| Metachromatic | Arylsulfatase A | 250100 | ARSA | 607574 | sulfatides |
| leukodystrophy | | | | | |

Other disorders of complex molecule degradation

| | | | | | |
|---|---|---|---|---|---|
| Niemann- | NPC intracellular | 257220, | NPC | 607623, | lysoSM, |
| Pick disease | cholesterol transporter | 607625 | | 601015 | lysoSM-509 |
| type C | | | | | |
| Lysosomal | Lysosomal acid lipase | 278000 | LIPA | 613497 | lysoSM-509 |
| acid lipase | | | | | oxysterols |
| deficiency | | | | | (non specific) |
| (LAL) | | | | | |

Disorders of peroxisomal fatty acid oxidation

| | | | | | |
|---|---|---|---|---|---|
| X-linked | ATP-binding cassette | 300100 | ABCD1 | 300371 | LPC26:0 |
| adrenoleukodystrophy | sub-family D member 1 | | | | VLCFA |

Peroxisomal biogenesis disorders

| | | | | | |
|---|---|---|---|---|---|
| Peroxin | Peroxisomal | 234580, | PEX | 602136, | LPC26:0 |
| deficiencies | biogenesis factors | 214100, | | 170993, | VLCFA |
| | | 601539, | | 603164, | Pristanic acid |
| | | 614866, | | 600414, | Phytanic acid |
| | | 614867, | | 601498, | DHCA |
| | | 617370, | | 603867, | THCA |
| | | 614882, | | 601758, | |
| | | 214110, | | 601789, | |
| | | 202370, | | 601791, | |
| | | 616716, | | 603360, | |
| | | 616617, | | 600279, | |
| | | 614862, | | 608666 | |
| | | 614863, | | | |
| | | 614920, | | | |
| | | 614859, | | | |
| | | 266510, | | | |
| | | 614883, | | | |
| | | 614885, | | | |
| | | 614887, | | | |
| | | 614876, | | | |
| | | 614877, | | | |
| | | 614886, | | | |
| | | 614872, | | | |
| | | 614873 | | | |

Disorders of copper metabolism

| | | | | | |
|---|---|---|---|---|---|
| MEDNIK | Adaptor related protein | 609313 | AP1S1 | 603531 | Copper |
| syndrome | complex 1 subunit | | | | Ceruloplasmin |
| | sigma 1 | | | | VLCFA |
| MEDNIK-like | Adaptor related protein | 242150 | AP1B1 | 600157 | Copper |
| syndrome | complex 1 subunit | | | | Ceruloplasmin |
| | beta 1 | | | | VLCFA |

In the last years, many treatable disorders have been included in newborn screening panels with the purpose to identify affected subjects before the onset of severe clinical manifestations.

The major issue of NBS is the relevant number of false positive results.

To reduce false positive rate, NBS algorithms include, when available, “second tier” testing.

In particular, NBS for neurodegenerative metabolic disorders is in mainly based on the determination of the individual enzyme activities (first tier testing) in DBS and, in positive cases, by the determination of the specific disease biomarkers in the same DBS sample (second tier testing). In this disease category, it is frequent the occurrence of enzymatic “pseudodeficiencies”, which causes a high rate of false positive results when first-tier testing relies on enzymatic activity determination. Therefore, for some diseases the first-tier testing is directly based on the biomarker determination in DBS.

Once the suspicion of NBS is confirmed, a diagnostic process is applied in the positive cases by referring the neonate to specialized centers/laboratories for further clinical, biochemical and molecular investigations.

Given the worldwide expansion of NBS programs, there is a growing need of improving the diagnostic capacities of NBS, in particular on the development of more sensitive and specific second-tier tests for DBS. Therefore, given the expansion of this diagnostic area, there is an urgent need to develop versatile and high throughput analytical methods, which can be applied in NBS, in confirmatory diagnostic tests, and in 20 monitoring of therapy.

Tandem mass spectrometry (MS/MS) technique is an adaptable and reliable tool that have found a wide application in biomarker discovery and clinical monitoring. Coupling it with liquid chromatography (LC) separation permits a rapid and simultaneous detection of different biomarkers in small amount, both in plasma or in dried blood spot (DBS).

The current LC-MS/MS methods for biomarker analysis of neurodegenerative metabolic disorders allow the quantification of different classes of biomarkers. Table 2 shows the most relevant published methods for biomarker analysis in selected neurodegenerative metabolic disorders.

TABLE 2

| | Matrix | | Biomarkers | | | | | |
|---|---|---|---|---|---|---|---|---|
| | Plasma | DBS | LysoGB3 | HexSph | LysoSM | Lyso509 | LysoGM1 | LysoGM2 |
| Menkovic | v | | | v | | | | |
| 2022 [4] | | | | | | | | |
| Malvagia | v | v | v | | | | | |
| 2021[5] | | | | | | | | |
| Hong | | v | | | | | | |
| 2021 [6] | | | | | | | | |
| Su | v | v | | | | | v | |
| 2021 [7] | | | | | | | | |
| Hong | | v | v | | v | | | |
| 2020 [8] | | | | | | | | |
| Polo | | v | v | v | v | v | v | v |
| 2019 [9] | | | | | | | | |
| Natarajan | | v | | | | | | |
| 2018 [10] | | | | | | | | |
| Pettazzoni | v | | v | v | v | v | v | v |
| 2017 [11] | | | | | | | | |
| Saville | v | | | | | | | |
| 2017 [12] | | | | | | | | |
| Polo | v | | v | v | v | v | | |
| 2016 [13] | | | | | | | | |
| Spacil | | v | | | | | | |
| 2016 [14 | | | | | | | | |
| Mirzaian | v | | | | | | | |
| 2015 [15] | | | | | | | | |
| Moyano | v | | | | | | | |
| 2013 [16] | | | | | | | | |

| | Biomarkers | | | | | | |
|---|---|---|---|---|---|---|---|
| | | | Sulfatide | Sulfatide | | | |
| | | Sulfatide | C16:0- | C16:1- | Sulfatide | | |
| | LPC26:0 | C16:0 | OH | OH | C18:0 | DHCA | THCA |
| Menkovic | | | | | | | |
| 2022 [4] | | | | | | | |
| Malvagia | | | | | | | |
| 2021[5] | | | | | | | |
| Hong | | v | | | | | |
| 2021 [6] | | | | | | | |
| Su | | | | | | | |
| 2021 [7] | | | | | | | |
| Hong | v | v | | | | | |
| 2020 [8] | | | | | | | |
| Polo | | | | | | | |
| 2019 [9] | | | | | | | |
| Natarajan | v | | | | | | |
| 2018 [10] | | | | | | | |
| Pettazzoni | | | | | | | |
| 2017 [11] | | | | | | | |
| Saville | | | | | v | | |
| 2017 [12] | | | | | | | |
| Polo | | | | | | | |
| 2016 [13] | | | | | | | |
| Spacil | | v | v | v | v | | |
| 2016 [14 | | | | | | | |
| Mirzaian | | v | v | | v | | |
| 2015 [15] | | | | | | | |
| Moyano | | v | | | v | | |
| 2013 [16] | | | | | | | |

However, as can be seen from Table 2, all known methods focus on single or on limited number of biomarkers, therefore not allowing the simultaneous evaluation of an exhausting number of neurodegenerative metabolic diseases.

In particular, some of the known methods shown in Table 2 are focused on a single target metabolite, such as GlcSph [4], LysoGB3 [5], LPC26:0 and C18-sulfatide [12], while some others provide simultaneous measurements of biomarker classes, such as lysosphingolipids and glycosphingolipids in plasma, DBS or, more rarely, in urine. As an example, two multiplex methods for the diagnosis of lysosphingolipidosis (biomarkers of Fabry disease, Gaucher disease, and Krabbe disease, ASMD, Niemann-Pick disease type C), GM1 and GM2 gangliosidosis, lack to analyse sulfatides, LPC26:0 and bile acids, the characteristic biormarkers of metachromatic leukodystrophy (MLD), X-linked adrenoleukodystrophy and Peroxisomal biogenesis disorders [9, 11].

As for MLD, sulfatides are the target diagnostic biomarkers and most of the published methods focus on a single analyte quantification or on a few selected sulfatides [12, 15, 16]. The most comprehensive method quantifies singles and total sulfatides in DBS and dried urine, needing enzymatic conversion and derivatization of sulfatides prior to the analysis, that results in a time-consuming process [14]. Finally, the most complete method, specifically designed for NBS, permits a 18-plex UHPLC-MS/MS assay for the screening of 15 lysosomal storage diseases, of X-linked adrenoleukodystrophy and some other errors of metabolism [8].

In the light of the above, it is therefore apparent the need to provide new diagnostic methods for neurodegenerative metabolic diseases, in particular newborn screening methods, able to overcome the disadvantages of the known methods.

According to the present invention a new method is provided for the simultaneous quantification in plasma and DBS of a large number of biomarkers, i.e. 13 biomarkers belonging to three different lipid classes (sphingolipids, lysophosphatidylcholines and sterol lipids), for the diagnosis of neurodegenerative metabolic diseases. In particular, according to the present invention, it has been surprisingly found that by using a particular extracting solution during the extraction step and specific analytic conditions during the detection step, it is possible to simultaneously extract and detect all said biomarkers.

More in detail, according to the present invention a new high throughput multiplex chromatography tandem mass spectrometry (UHPLC-MS/MS) method has been developed for the simultaneous quantification in plasma and DBS of a large number of biomarkers characteristic of neurodegenerative metabolic diseases: LysoHexSph (GalSph+GlcSph), LysoGb3, LysoSM, LysoSM-509, LysoGM1, LysoGM2, lysophosphatidylcholine LPC26:0, THCA and DHCA bile acids, and C16-, C18-, C16:1-OH-, C16-OH-sulfatide species.

Compared to the existing methods listed in Table 2, the method according to the invention advantageously provides a great innovation, since it allows the simultaneous analysis for a large number, i.e. 13, of neurodegenerative metabolic diseases, including lysosphingolipidosis [Fabry disease, Gaucher disease, and Krabbe disease, ASMD (former Niemann Pick disease type A/B), Niemann-Pick disease type C (NPC)], GM1 gangliosisodis, GM2 gangliosidosis, metachromatic leukodystrophy, and X-linked adrenoleukodistrophy.

Furthermore, biomarker analysis according to the present invention, advantageously allows the detection of peroxisomal biogenesis disorders and of two rare inherited diseases of copper metabolism (MEDNIK and MEDNIK-like syndromes). Moreover, the simultaneous analysis of such extended number of analytes permitted the identification of new biomarkers for Krabbe, Gaucher and MEDNIK syndrome.

Table 3 reported below shows a comparison of the method of the present invention with the known methods listed in Table 2, whereas Table 4 shows a comparison of the method of the invention with Hong et al, methods [8].

TABLE 3

| | Matrix | | Biomarkers | | | | | |
|---|---|---|---|---|---|---|---|---|
| | Plasma | DBS | LysoGB3 | HexSph | LysoSM | Lyso509 | LysoGM1 | LysoGM2 |
| Method | v | v | v | v | v | v | v | v |
| of the | | | | | | | | |
| present | | | | | | | | |
| invention | | | | | | | | |

Published methods

| | | | | | | | | |
|---|---|---|---|---|---|---|---|---|
| Menkovic | v | | | v | | | | |
| 2022 | | | | | | | | |
| Malvagia | v | v | v | | | | | |
| 2021 | | | | | | | | |
| Hong | | v | | | | | | |
| 2021 | | | | | | | | |
| Su | v | v | | | | | v | |
| 2021 | | | | | | | | |
| Hong | | v | v | | v | | | |
| 2020 | | | | | | | | |
| Polo | | v | v | v | v | v | v | v |
| 2019 | | | | | | | | |
| Natarajan | | v | | | | | | |
| 2018 | | | | | | | | |
| Pettazzoni | v | | v | v | v | v | v | v |
| 2017 | | | | | | | | |
| Saville | v | | | | | | | |
| 2017 | | | | | | | | |
| Polo | v | | v | v | v | v | | |
| 2017 | | | | | | | | |
| Spacil | | v | | | | | | |
| 2016 | | | | | | | | |
| Mirzaian | v | | | | | | | |
| 2015 | | | | | | | | |
| Moyano | v | | | | | | | |
| 2013 | | | | | | | | |

| | Biomarkers | | | | | | |
|---|---|---|---|---|---|---|---|
| | | | Sulfatide | Sulfatide | | | |
| | | Sulfatide | C16:0- | C16:1- | Sulfatide | | |
| | LPC26:0 | C16:0 | OH | OH | C18:0 | DHCA | THCA |
| Method | v | v | v | v | v | v | v |
| of the | | | | | | | |
| present | | | | | | | |
| invention | | | | | | | |
| | Published methods | | | | | | |
| Menkovic | | | | | | | |
| 2022 | | | | | | | |
| Malvagia | | | | | | | |
| 2021 | | | | | | | |
| Hong | | v | | | | | |
| 2021 | | | | | | | |
| Su | | | | | | | |
| 2021 | | | | | | | |
| Hong | v | v | | | | | |
| 2020 | | | | | | | |
| Polo | | | | | | | |
| 2019 | | | | | | | |
| Natarajan | v | | | | | | |
| 2018 | | | | | | | |
| Pettazzoni | | | | | | | |
| 2017 | | | | | | | |
| Saville | | | | | v | | |
| 2017 | | | | | | | |
| Polo | | | | | | | |
| 2017 | | | | | | | |
| Spacil | | v | v | v | v | | |
| 2016 | | | | | | | |
| Mirzaian | | v | v | | v | | |
| 2015 | | | | | | | |
| Moyano | | v | | | v | | |
| 2013 | | | | | | | |

TABLE 4

| | Method of the present | | Hong et al. | |
|---|---|---|---|---|
| | invention | | 2020 ref. 8 | |
| | DBS | Plasma | DBS | DBS |
| | biomarker | biomarker | enzyme | biomarker |
| Fabry | v | v | v | v |
| Gaucher | v | v | v | |
| Krabbe | v | v | v | |
| ASMD | v | v | v | v |
| LALD | | | v | |
| (Wolman) | | | | |
| MLD | v | v | | v |
| X-ALD | v | v | | v |
| Niemann- | v | v | | |
| Pick type C | | | | |
| GM1 | | v | | |
| GM2 | | v | | |
| MPS I | | | v | |
| MPS II | | | v | |
| MPS IIIb | | | v | |
| MPS Iva | | | v | |
| MPS VI | | | v | |
| MPS VII | | | v | |
| Pompe | | | v | |
| NCL 1 | | | v | |
| NCL 2 | | | v | |
| Galactosemia | | | v | |
| (GALT) | | | | |
| Biotinidase | | | v | |
| MEDNIK & | v | v | | |
| MEDNIK-like | | | | |

As shown in Table 4, the known LC-MS/MS method relies on the combined analysis of enzymatic activities with biormakers quantification. In particular, the 14 enzymatic activity measurements require seven parallel extractions/incubations and a complex sample preparation that leads to an increment of time and costs for the analysis.

The LC-MS/MS method according to the present invention, compared to the one described by Hong et al [8], allows a most extended number of biomarkers measurable in a single run, with a simpler and faster sample preparation, providing several advantages if compared to the more complex and time-consuming enzyme assay combined with biomarker quantification.

According to the present invention, quantification of biomarkers with good precision and accuracy permitted to determine physiological and pathological concentration ranges in plasma and DBS.

The method according to the invention does not separate the two isomers of LysoHexSph [LysoGalSph (charcteristc of Gaucher disease) and LysoGlcSph (characteristic of Krabbe disease)], however the two diseases can be distinguished taking into consideration that: 1) the absolute values of the LysoHexSph are significantly higher in Krabbe than in Gaucher, 2) LysoGB3, is elevated in Gaucher only, and 3) C18-sulfatide was significantly higher in Krabbe disease only. Therefore, by applying the method of the present invention, there is no need to further investigate which specific isomer of LysoHexSph is present in the sample, as done in prior art methods, because the simultaneous detection of LysoGB3 and of C18-sulfatide according to the invention advantageously allows to discriminate between Gaucher and Krabbe disease, respectively.

On the basis of the results reported below, according to the method of the present invention, analysis of LysoSM-509 (also named Lyso509 below) and LysoSM in plasma permits a clear differentiation between ASMD and Niemann Pick disease type C based on a quantitative evaluation: with respect to healthy patients and patients with NPC or LAL, LysoSM is significantly higher in ASMD and the ratio Lyso509/LysoSM is higher in Niemann Pick type C and not in ASMD. DBS can be utilized for ASMD screening, due to the significant elevation of both LysoSM and Lyso509, whereas NPC patients had only slightly elevated levels of Lyso509 and normal levels of LysoSM.

It was observed that Lyso509 was elevated also in LAL, and a mild elevation was seen in single patients with GM1-gangliosidosis and peroxisomal disorders.

Moreover, LPC26:0 was seen as a reliable and selective biomarker of X-linked adrenoleukodistrophy and Peroxisomal biogenesis disorders. However, by measuring LPC26:0 alone, the discrimination between X-ALD and PBD patients is not possible, since both conditions present elevation of LPC26:0 and different analyses are needed. The advantage of the method of the present invention relies on the simultaneous analysis of both LPC26:0 and bile acids (DHCA and THCA, present only in PBD), that permits the distinction between X-ALD and PBD patients, without the need of further laboratory investigations.

The method according to the invention as applied in the experiments described below included the analysis of four sulfatides as MLD biomarkers. The selection of these four species was based on a preliminary study performed on an extended number of sulfatides in MLD and control samples. As already reported [13], the hydroxylated forms C16:1-OH- and C16-OH-sulfatides are the best discriminant biomarkers for MLD, both in plasma and DBS. Moreover, the analysis of 2 additional sulfatides (C18- and C16-sulfatides) may further confirm the diagnosis of MLD, and were identified as novel biormakers for Krabbe disease (C18-sulfatide) and for MEDNIK and MEDNIK-like diseases (C16-sulfatide), never reported so far.

Finally, according to the present invention, a number of samples from patients with other neurometabolic diseases was analysed, including Menkes disease, Kearn-Sayre syndrome, Smith-Lemli-Opitz and Chanarin Dorfman diseases, cerebrotendinous xanthomatosis, Vici syndrome, mucolipidosis type II, WDR45 mutation, methylmalonic and propionic acidemias, without observing elevation of tested biomarkers.

The method according to the present inventions can be advantageously used for the following applications: NBS (first or second tier testing in DBS), selective screening in patients with neurodegenerative diseases (plasma/DBS), confirmatory testing for genetic panels of neurodegenarive diseases (plasma/DBS) and treatment monitoring by biomarker analysis (plasma/DBS).

In addition, the method according to the present invention could be advantageously used also for pre-natal diagnosis by applying the method on amniotic fluid samples. The experimental data reported below show the method of the invention applied on plasma and DBS samples. It is plausible for a person skilled in the art that, when the biological sample is amniotic fluid, the extraction step can be carried out with the same extracting solution used for plasma, since plasma and amniotic fluid share several major components.

It is therefore specific object of the present invention a method for the in vitro diagnosis of one or more of neurodegenerative metabolic diseases selected from the group consisting of
lysosphingolipidosis chosen from Fabry disease, Gaucher disease, Krabbe disease, Acid sphingomyelinase deficiency (ASMD, also intended as Niemann Pick disease type A or B), Niemann-Pick disease type C (NPC); GM1 gangliosisodis; GM2 gangliosidosis; lysosomal acid lipase deficiency (LALD, also known as Wolman disease); metachromatic leukodystrophy (MLD); X-linked adrenoleukodystrophy (X-ALD) and its phenotypic variant Adrenomyeloneuropathy (AMN); MEDNIK disease; MEDNIK-like disease; peroxisomal biogenesis disorders (PBD);
said method comprising or consisting of
a) contacting a biological sample of a subject with an extracting solution able to extract all the following biomarkers: lysoGM1, lysoGM2, lysoGB3, lysoSM, lysoSM-509, lysoHexSph (which is composed by the two isomers lysoGalSph and lysoGlcSph), LPC26:0, Sulfatide C18:0 (C18-sulfa), Sulfatide C16:0 (C16-sulfa), Sulfatide C16:1-OH (C16:1-OH), Sulfatide C16:0-OH (C16-OH), DHCA, THCA, said biological sample being plasma, dried blood spot (DBS) and/or amniotic fluid, preferably plasma, in order to obtain an extracted solution;
b) detecting the possible presence of one or more of said biomarkers in said extracted solution by means of liquid chromatography, preferably ultra-high performance liquid chromatography (UPLC), combined to mass spectrometry, preferably tandem mass spectrometry (MS/MS);
wherein, when said biological sample is plasma or amniotic fluid, said extracting solution comprises or consists of
C1-C3 alcohol, such as methanol (CH₃OH), in a percentage from 55% to 65%, preferably 60%;
C3-C6 ketone, such as acetone (C₃H₆O), in a percentage from 25% to 35%, preferably 30%;
water, in a percentage from 5% to 15%, preferably 10%; whereas
when said biological sample is DBS, said extracting solution comprises or consists of C1-C3 alcohol, such as methanol (CH₃OH) in a percentage from 90% to 100%; when the percentage is from 90% to less than 100%, the solution of C1-C3 alcohol is in water and/or in one or more organic solvents, such as other C1-C3 alcohols or acetonitrile;
wherein said liquid chromatography is carried out by using a C6-C18 reverse phase column, for example a C6-C10 reverse phase column, such as High Strength Silica T3 C18 column, Ethylene Bridged Hybrid C18 column, phenyl column, Ethylene Bridged Hybrid C18 phenyl column, Core Shell Mode phases column, preferably a C6-C8 reverse phase column with phenyl groups bound to the silica surface; and
a mobile phase comprising an organic solvent, such as acetonitrile, methanol, ethanol or 2-propanol, preferably acetonitrile, and an aqueous solvent, preferably water;
wherein said mass spectrometry is carried out both in positive and negative ionization modes, where LysoGM1, LysoGB3, Lyso509, LysoSM, LysoHexSph, LPC26:0, LysoGM2 and LPC26:0 are detected in positive ionization mode, C18-sulfatide, C16-sulfatide, C16:1-OH-sulfatide and C16-OH-sulfatide are detected in negative ionization mode and DHCA and THCA are detected in positive ionization mode or in negative ionization mode, preferably in negative ionization mode;
and wherein
lysoGM1 is for the diagnosis of GM1 gangliosisodis;
lysoGM2 is for the diagnosis of GM2 gangliosidosis;
lysoGB3 is for the diagnosis of Fabry disease;
lysoSM and lysoSM-509 are for the diagnosis of Acid sphingomyelinase deficiency and Niemann-Pick disease type C;
lysoSM-509 is for the indication of possible lysosomal acid lipase deficiency;
HexSph is for the diagnosis of Gaucher and Krabbe diseases;
LPC26:0 is for the diagnosis of X-linked adrenoleukodystrophy and peroxisomal biogenesis disorders
Sulfatide C18:0 is for the diagnosis of leukodystrophy and Krabbe disease;
Sulfatide C16:0 is for the diagnosis of Metachromatic leukodystrophy, MEDNIK and MEDNIK-like syndrome;
Sulfatide C16:1-OH is for the diagnosis of Metachromatic leukodystrophy;
Sulfatide C16:0-OH is for the diagnosis of Metachromatic leukodystrophy;
DHCA is for the diagnosis of peroxisomal biogenesis disorders;
THCA is for the diagnosis of peroxisomal biogenesis disorders.

According to the method of the present invention
GM1 gangliosidosis is diagnosed when the presence of GM1 biomarker is detected in the biological sample;
GM2 gangliosidosis is diagnosed when the presence of GM2 biomarker is detected in the biological sample;
Fabry disease is diagnosed when the concentration of lysoGB3 biomarker detected in the biological sample is higher than the concentration of lysoGB3 biomarker detected in a biological sample of a healthy subject with the same method;
acid sphingomyelinase deficiency and Niemann-Pick disease type C are diagnosed when the concentration of both lysoSM and lysoSM-509 biomarkers detected in the biological sample is higher than the concentration of lysoSM and lysoSM-509 biomarkers detected in a biological sample of a healthy subject with the same method; in particular, it is known that is possible to distinguish between acid sphingomyelinase deficiency and Niemann-Pick disease type C on the basis of the levels of expression of lysoSM-509 and lysoSM and on the basis of their ratio; specifically, when acid sphingomyelinase deficiency is present the ratio of the concentrations LysoSM-509/LysoSM˜1 while when Niemann-Pick disease type C is present the ratio of the concentrations LysoSM-509/LysoSM>3;
lysosomal acid lipase deficiency (LALD) can be suspected and, therefore, can be considered in the differential diagnosis, when lysoSM-509 is higher than the concentration of lysoSM-509 biomarker detected in a biological sample of a healthy subject with the same method;
Krabbe disease is diagnosed when the concentration of both LysoHexSph and Sulfatide C18:0 biomarkers detected in the biological sample is higher than the concentration of LysoHexSph and Sulfatide C18:0 biomarkers detected in a biological sample of a healthy subject with the same method;
Gaucher disease is diagnosed when the concentration of LysoHexSph biomarker detected in the biological sample is higher than the concentration of LysoHexSph biomarker detected in a biological sample of a healthy subject with the same method and when C18:0 biomarker detected in the biological sample is not higher than the concentration of Sulfatide C18:0 biomarkers detected in a biological sample of a healthy subject with the same method;
peroxisomal biogenesis disorders are diagnosed when the concentration of LPC26:0 biomarker detected in the biological sample is higher than the concentration of LPC26:0 biomarker detected in a biological sample of a healthy subject with the same method and when DHCA and THCA are present in the biological sample;
X-linked adrenoleukodystrophy and its phenotypic variant Adrenomyeloneuropathy (AMN) are diagnosed when the concentration of LPC26:0 biomarker detected in the biological sample is higher than the concentration of LPC26:0 biomarker detected in a biological sample of a healthy subject with the same method and when DHCA and THCA are absent in the biological sample;
metachromatic leukodystrophy is diagnosed when the concentration of Sulfatide C18:0, Sulfatide C16:0, Sulfatide C16:1-OH and Sulfatide C16:0-OH biomarkers detected in the biological sample is higher than the concentration of Sulfatide C18:0, Sulfatide C16:0, Sulfatide C16:1-OH and Sulfatide C16:0-OH biomarkers detected in a biological sample of a healthy subject with the same method;
MEDNIK or MEDNIK-like syndrome are diagnosed when the concentration of Sulfatide C16:0 biomarker detected in the biological sample is higher than the concentration of Sulfatide C16:0 biomarker detected in a biological sample of a healthy subject with the same method.

Therefore, the present invention concerns a method for the simultaneous detection and quantification of lysoGM1, lysoGM2, lysoGB3, lysoSM, lysoSM-509, lysoHexSph, LPC26:0, Sulfatide C18:0, Sulfatide C16:0, Sulfatide C16:1-OH, Sulfatide C16:0-OH, DHCA and THCA biomarkers in a biological sample of a subject, said method comprising or consisting of
a) contacting said biological sample with an extracting solution able to extract all the following biomarkers: lysoGM1, lysoGM2, lysoGB3, lysoSM, lysoSM-509, lysoHexSph (which is composed by the two isomers LysoGalSph and LysoGlcSph), LPC26:0, Sulfatide C18:0 (C18-sulfa), Sulfatide C16:0 (C16-sulfa), Sulfatide C16:1-OH (C16:1-OH), Sulfatide C16:0-OH (C16-OH), DHCA, THCA, said biological sample being plasma, dried blood spot and/or amniotic fluid, preferably plasma, in order to obtain an extracted solution;
b) detecting the possible presence of one or more of said biomarkers in said extracted solution by means of liquid chromatography, preferably ultra-high performance liquid chromatography (UPLC), combined to mass spectrometry, preferably tandem mass spectrometry (MS/MS);
wherein, when said biological sample is plasma or amniotic fluid, said extracting solution comprises or consists of
C1-C3 alcohol, such as methanol (CH₃OH), in a percentage from 55% to 65%, preferably 60%;
C3-C6 ketone, such as acetone (C₃H₆O), in a percentage from 25% to 35%, preferably 30%;
water, in a percentage from 5% to 15%, preferably 10%; whereas
when said biological sample is dried blood spot, said extracting solution comprises or consists of C1-C3 alcohol, such as methanol (CH₃OH), in a percentage from 90% to 100%; when the percentage is from 90% to less than 100%, the solution of C1-C3 alcohol is in water and/or in one or more organic solvents, such as other C1-C3 alcohols or acetonitrile;
wherein said liquid chromatography is carried out by using
a C6-C18 reverse phase column, for example a C6-C10 reverse phase column, such as High Strength Silica T3 C18 column, Ethylene Bridged Hybrid C18 column, phenyl column, Ethylene Bridged Hybrid C18 phenyl column, Core Shell Mode phases column, preferably a C6-C8 reverse phase column with phenyl groups bound to the silica surface; and
a mobile phase comprising an organic solvent, such as acetonitrile, methanol, ethanol or 2-propanol, preferably acetonitrile, and an aqueous solvent, preferably water;
wherein said mass spectrometry is carried out both in positive and negative ionization modes, wherein LysoGM1, LysoGB3, Lyso509, LysoSM, LysoHexSph, LPC26:0, LysoGM2 and LPC26:0 are detected in positive ionization mode, C18-sulfatide, C16-sulfatide, C16:1-OH-sulfatide and C16-OH-sulfatide are detected in negative ionization mode and DHCA and THCA are detected in positive ionization mode or in negative ionization mode, preferably in negative ionization mode.

According to the present invention, said mobile phase can further comprise a buffer solution comprising
An ammonium salt, such as ammonium formate, ammonium acetate, preferably ammonium formate, in an amount ranging from 5 to 20 mM;
a weak organic acid, such as formic acid, acetic acid, trifluoroacetic or perfluoroeptanoic acid, preferably formic acid, in an amount ranging from 0.1 to 5%; and
an aqueous solvent, such as water.

For example, said buffer solution can comprise ammonium formate in concentration of 10 mM and formic acid at a percentage of 0.1% in aqueous solvent.

According to the present invention, said mobile phase can comprise or consist of one or more separate mobile phases, for example a first and a second mobile phase, which can be mixed together at different ratios in order to obtain the mobile phase according to the method of the invention.

According to an embodiment of the present invention, said mobile phase can comprise or consist of
a first phase comprising an organic solvent, such as acetonitrile, methanol, ethanol or 2-propanol, preferably acetonitrile, at a percentage of 40%, in aqueous solvent, preferably water;
a second phase comprising said organic solvent at a percentage of 95%, in said aqueous solvent.

Said first phase and said second phase can further comprise a buffer solution comprising
an ammonium salt, such as ammonium formate, ammonium acetate, preferably ammonium formate, in an amount ranging from 5 to 20 mM;
a weak organic acid, such as formic acid, acetic acid, trifluoroacetic or perfluoroeptanoic acid, preferably formic acid, in an amount ranging from 0.1 to 5%; and
an aqueous solvent, such as water.

For example, said first and second phases can comprise a buffer solution comprising ammonium formate in concentration of 10 mM and formic acid at a percentage of 0.1% in aqueous solvent.

According to an embodiment of the present invention, said liquid chromatography can be carried out using C6-Phenyl 100×5 mm column with a 3 μm particle size maintained at 50° C. and 0.6 ml/min flow, preferably with a gradient of mobile phases as reported in Table 5 below.

According to the method of the invention, said liquid chromatography combined with mass spectrometry carried out both in positive and negative ionization modes can be carried out using an electrospray ionization source, preferably settled at 550° C. and in scheduled acquisition mode.

According to the present invention, said method can be carried out on pre-natal, neo-natal or post-natal biological sample.

For pre-natal biological sample is intended a sample of amniotic fluid. For neo-natal biological sample is intended a sample of plasma or DBS collected from the subject within 7 days from birth. For post-natal biological sample is intended a sample of plasma or DBS collected from the subject after 7 days from birth, for example from an adult subject. Therefore, the diagnosis according to the present invention can be a pre-natal, neo-natal or post-natal diagnosis or screening.

The present invention concerns also a kit, for example a kit for carrying out the method of diagnosis or the method of simultaneous detection as defined above, for the in vitro diagnosis of one or more neurodegenerative diseases selected from the group consisting of
lysosphingolipidosis chosen from Fabry disease, Gaucher disease, Krabbe disease, Acid sphingomyelinase deficiency (ASMD, also intended as Niemann Pick disease type A or B), Niemann-Pick disease type C (NPC); GM1 gangliosisodis; GM2 gangliosidosis; lysosomal acid lipase deficiency (LALD, also known as Wolman disease); metachromatic leukodystrophy (MLD); X-linked adrenoleukodystrophy (X-ALD) and its phenotypic variant Adrenomyeloneuropathy (AMN); MEDNIK disease; MEDNIK-like disease; peroxisomal biogenesis disorders (PBD);
said kit comprising or consisting of
i) an extraction solution comprising or consisting of
C1-C3 alcohol, such as methanol (CH₃OH), in a percentage from 55% to 65%, preferably 60%;
C3-C6 ketone, such as acetone (C₃H₆O), in a percentage from 25% to 35%, preferably 30%;
water, in a percentage from 5% to 15%, preferably 10%;
and/or an extracting solution comprising or consisting of C1-C3 alcohol, such as methanol (CH₃OH) in a percentage from 90% to 100% (when the percentage is from 90% to less than 100%, the solution of C1-C3 alcohol is in water and/or in one or more organic solvents, such as other C1-C3 alcohols or acetonitrile);
ii) a mobile phase comprising an organic solvent, such as acetonitrile, methanol, ethanol or 2-propanol, preferably acetonitrile, and an aqueous solvent or suitable ingredients able to provide said mobile phase when mixed with an aqueous solvent, such as formic acid, ammonium acetate, ammonium formate, acetic acid, trifluoroacetic and perfluoroeptanoic acid, preferably formic acid.
iii) a C6-C18 reverse phase column, for example a C6-C10 reverse phase column, such as High Strength Silica T3 C18 column, Ethylene Bridged Hybrid C18 column, phenyl column, Ethylene Bridged Hybrid C18 phenyl column, Core Shell Mode phases column, preferably a C6-C8 reverse phase column with phenyl groups bound to the silica surface;

According to the present invention, said kit can further comprise one or more pooled samples, such as one or more pooled samples of plasma, added with standard concentrations of biomarkers, for example one or more pooled samples added with a standard mixture of biomarkers for the calibration or one or more pooled samples added with two different concentrations of a biomarker as control.

According to the present invention, said kit can further comprise a mixture of internal standards.

According to the present invention, said kit can further comprise a buffer solution comprising
an ammonium salt, such as ammonium formate, ammonium acetate, preferably ammonium formate, in an amount ranging from 5 to 20 mM;
a weak organic acid, such as formic acid, acetic acid, trifluoroacetic and perfluoroeptanoic acid, preferably formic acid, in an amount ranging from 0.1 to 5%; and
an aqueous solvent, such as water.

For example, in the kit according to the invention, said buffer solution can comprise ammonium formate in concentration of 10 mM and formic acid at a percentage of 0.1% in aqueous solvent.

According to the kit of the present invention, the mobile phase can comprise or consist of one or more separate mobile phases, for example a first and a second mobile phase, which can be mixed together at different ratios in order to obtain the final mobile phase. For example, the mobile phase can comprise or consist of
a first phase comprising an organic solvent, such as acetonitrile, methanol, ethanol or 2-propanol, preferably acetonitrile, at a percentage of 40%, in aqueous solvent, preferably water;
a second phase comprising said organic solvent at a percentage of 95% in said aqueous solvent.

The present invention also concerns a method for the in vitro diagnosis of Krabbe disease, said method comprising detecting in a biological sample of a subject sulfatide C18 and LysoHexSph,
wherein a higher concentration of sulfatide C18 and LysoHexSph in said biological sample with respect to the concentration of sulfatide C18 and LysoHexSph in a biological sample of a healthy subject indicates Krabbe disease, said biological sample being plasma, dried blood and/or amniotic fluid.

The present invention also concerns the use of sulfatide C18 as biomarker for the in vitro diagnosis of Krabbe disease, which can be used also in combination with LysoHexSph as biomarkers for the in vitro diagnosis of Krabbe disease.

The present invention also concerns a method for the in vitro diagnosis of MEDNIK or MEDNIK-like diseases, said method comprising detecting in a biological sample of a patient sulfatide C16,
wherein a higher concentration of sulfatide C16 in said biological sample with respect to the concentration of sulfatide C16 in a biological sample of a healthy subject indicates MEDNIK or MEDNIK-like diseases, said biological sample being plasma, dried blood and/or amniotic fluid.

The present invention also concerns the use of sulfatide C16 as biomarker for the in vitro diagnosis of MEDNIK or MEDNIK-like diseases.

The present invention now will be described by an illustrative, but not limitative way, according to preferred embodiments thereof, with particular reference to the drawings and the example below, wherein

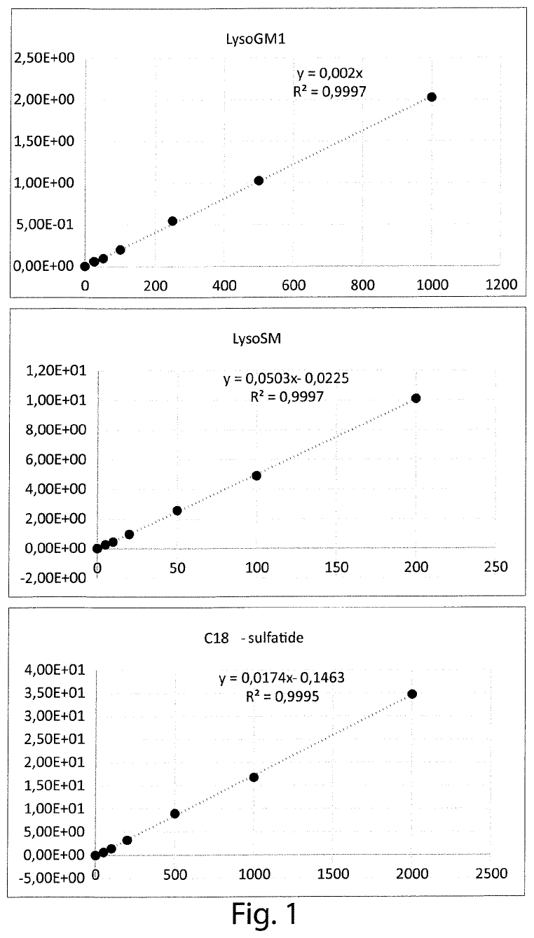

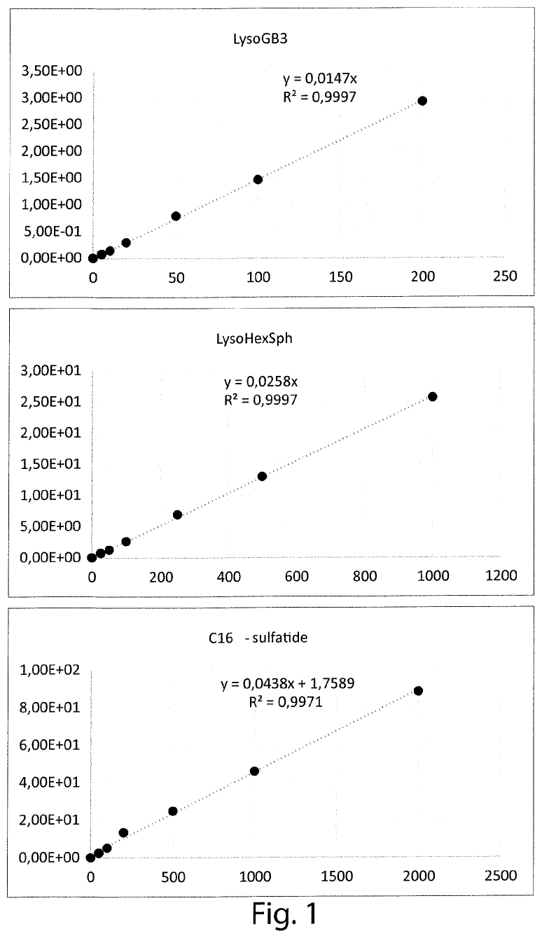

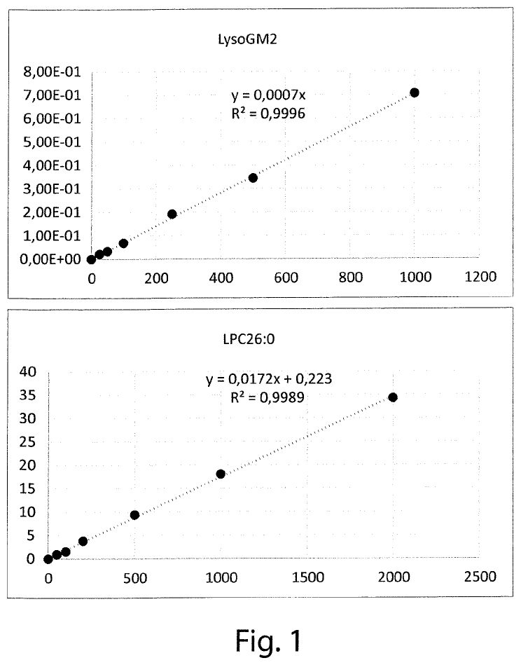

FIG. 1 shows calibration curves of analytes obtained from spiked human plasma; and

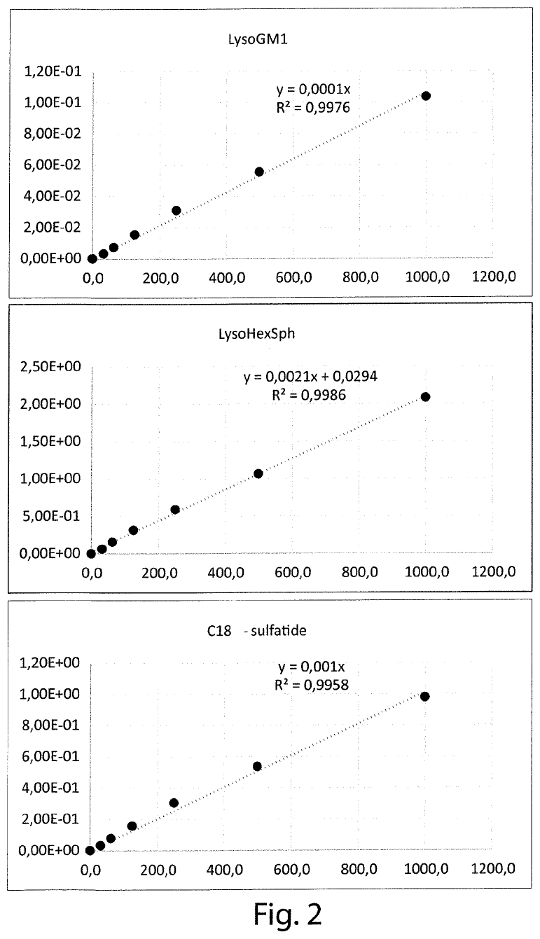

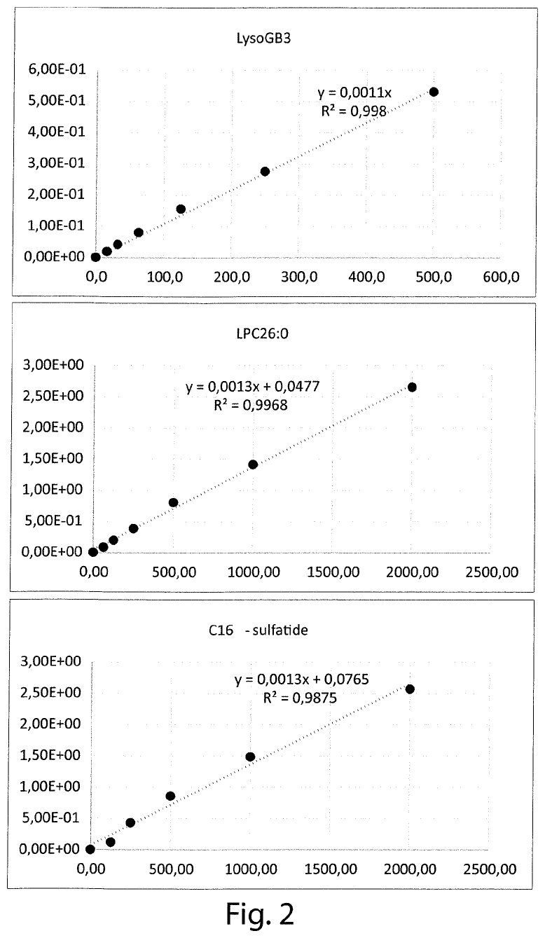

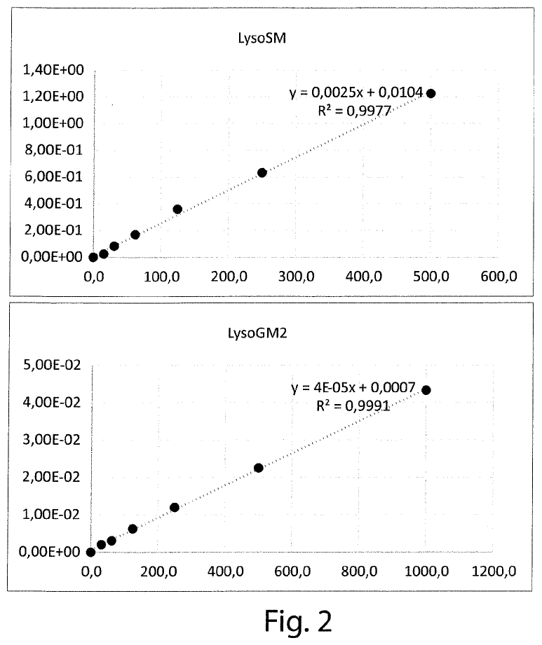

FIG. 2 shows calibration curves of analytes obtained from spiked DBS.

### EXAMPLE 1: DEVELOPMENT AND VALIDATION OF THE METHOD FOR THE DIAGNOSIS OF NEURODEGENERATIVE METABOLIC DISEASES ACCORDING TO THE PRESENT INVENTION

### Materials and Methods

### Chemicals and Reagents

The lipid standards 1-beta-D-glucosylsphingosine (LysoGlcSph), lyso-globotriaosylsphingosine (LysoGb3), lyso-Sphingomyelin (LysoSM), lyso-sphingomyelin-D7 (LysoSM-D7), LysoGM1 were purchased from Sigma-Aldrich/MERCK (Burlington, MA, USA [). 1-hexacosanoyl-2-hydroxy-sn-glycero-3-phosphocholine (LPC 26:0), 1-hexacosanoyl-d4-2-hydroxy-sn-glycero-3-phosphocholine (LPC 26:0-D4) were instead purchased from Avanti Polar Lipids (Alabaster, AL, USA). Lyso-monosialoganglioside GM₂-NH₄_{⁺} salt (LysoGM2), N-Hexadecanoyl-sulfatide (C16-sulfatide), N-Octadecanoyl-sulfatide (C18-sulfatide), N-omega-CD₃-Octadecanoyl-sulfatide (C18-D3-sulfatide) were bought from Matreya LLC (State College, PA, USA). 3α,7α-dihydroxy-5β-cholestan-26-oic acid (DHCA), 3α,7α,12α-trihydroxy-5β-cholestan-26-oic acid (THCA), 27,27,27 [D₃]-3α,7α-dihydroxy-5β-cholestan-26-oic acid (DHCA-D3), 27,27,27-[D₃]-3α,7α,12α-trihydroxy-5β-cholestan-26-oic acid (THCA-D3) were purchased from VU Medical Center Metabolic Laboratory (Amsterdam, The Netherlands).

Acetonitrile, methanol (HPLC-MS-gradient grade) and chloroform were purchased from Sigma Aldrich (St Louis, MO, USA). The ULC-MS-grade 99% formic acid was supplied by Biosolve Chimie (Dieuze, France). The laboratory reagent grade acetone was purchased from Fisher Scientific UK Ltd (Loughborough, UK). Ultrapure water was generated using the Milli-Q system (Millipore, Bedford, MA, USA). The stock standard solutions for all analytes were prepared by solving the powder materials in the mix of methanol and chloroform as recommended by manufacturers. Ulterior dilutions were made with pure methanol and aliquots were stored at −80° C. before use.

### Patient Samples

The experimental protocol was reviewed and approved by the Ethical Committee of Bambino Gesù Children's Hospital IRCC (2119_OPBG_2020). The studies were performed in accordance with the Declaration of Helsinki and informed consents were obtained from all patients/parents.

The plasma and DBS samples were obtained from patients followed at the Metabolic Diseases Unit of Bambino Gesù Children's Hospital. The age range of patients varied from 0.1 to 72 years (median 9.7 years). To establish normal reference values of biomarkers, control samples were taken from 122 (plasma) and 188 (DBS) anonymous healthy blood donors with age range of 0.1-63 years (median 11.6 years). Plasma and DBS samples from patients with Fabry diseases, Gaucher diseases, Krabbe diseases, ASMD, Niemann-Pick disease type C, Wolman/CESD (LAL), metachromatic leukodystrophy (MLD), GM1-e GM2-gangliosidosis, peroxisomial diseases (X-ALD/AMN, PBD) e cell trafficking diseases (MEDNIK, MEDNIK-like) were collected during scheduled clinical monitoring. All patients had a confirmed diagnosis based on enzyme and/or molecular analysis. Plasma samples were collected in EDTA-containing tubes. After 5 minutes of centrifugation plasma was separated and stored frozen at −80° C. in the OPBG's Bio-bank until analysis. DBS samples were stored at room temperature after collection.

### Calibration Standards and Quality Control (QC) Preparation

### Plasma

The internal standard solution was prepared as the mix of LysoSM-D7 (50 nM), LPC26:0-D4 (1 μM), C18-D3-sulfatide (1 μM), DHCA-D3 (5 μM), and THCA-D3 (5 M) in methanol. The extraction solution functioning also for plasma protein precipitation was composed of methanol, acetone and water (60:30:10).

For plasma analysis seven-point calibration curves were prepared by spiking the pure standards with pooled human plasma with following concentration ranges: 0-200 nM for LysoGb3; 0-1000 nM for LysoGM1, LysoGM2, LysoSM, LysoHexSph and C18-sulfatide; 0-2000 nM for LPC26:0 and C16-sulfatide. Aliquots of standards in methanol were placed in a glass tube and dried under nitrogen stream, then a corresponding amount of plasma was added to obtain the highest concentration point of calibration curve. The glass tube with plasma was mixed on thermomixer for over 23 hours at 37° C. and 450 rpm to achieve a homogeneous and complete solubilisation of standards. The highest calibration point was further diluted with pooled plasma to obtain other calibration points.

The precision and accuracy of the assay were evaluated using the QCs at three concentration levels, which were prepared from pooled plasma added with pure standards as follows: for LysoGb3 QC1 (endogenous), QC2 (endogenous+2 nmol/L), QC3 (endogenous+20 nmol/L); for LysoGM1, LysoGM2, LysoHexSph and LysoSM: QC1 (endogenous), QC2 (endogenous+10 nmol/L), QC3 (endogenous+100 nmol/L); for C18-sulfatide: QC1 (endogenous), QC2 (endogenous+20 nmol/L), QC3 (endogenous+200 nmol/L); for LPC 26:0 and C16-sulfatide QC1 (endogenous), QC2 (endogenous+200 nmol/L), QC3 (endogenous+2000 nmol/L). First, the QC3 was prepared as described for the highest point of calibration curve, then QC2 was prepared from QC3 by dilution with pooled plasma.

### DBS

The internal standard solution functioning also as extraction solution was prepared as the mix of LysoSM-D7 (2.5 nM), LPC26:0-D4 (50 nM), C18-D3-sulfatide (50 nM), DHCA-D3 (500 nM), and THCA-D3 (500 nM) in methanol. Seven-point calibration curves were prepared with following concentration ranges: 0-500 nM for LysoGb3 and LysoSM; 0-1000 nM for LysoGM1, LysoGM2, LysoHexSph and C18-sulfatide; 0-2000 nM for LPC26:0 and C16-sulfatide. The preparation of calibration curve points was as follows: 50 μL of human blood was dropped on the DBS collection card to fill the printed 13 mm diameter circle. After blood spots had dried (about 3 hours on air) 25 μL of standard in methanol was dropped on DBS and left on air for complete methanol evaporation. The resulting DBS standard concentration was assumed as 2-fold reduced original concentration in methanol solution.

The preparation of QCs for accuracy and precision assay followed the same procedures as preparation of calibration curve points. The concentration was: for LysoGb3 and LysoSM-QC1 (endogenous), QC2 (endogenous+100 nmol/L), QC3 (endogenous+200 nmol/L); for LysoGM1, LysoGM2, LysoHexSph, C18-, C16-sulfatides and LPC26:0-QC1 (endogenous), QC2 (endogenous+250 nmol/L), QC3 (endogenous+500 nmol/L).

In all experiments LysoGlcSph was used as the LysoHexSph standard. Due to of the lack of commercial standards the measurement of LysoSM-509, and sulfatide species C16-OH and C16:1-OH was performed using Multiple of Median (MOM) calculations. The indicative concentrations of bile acids−DHCA and THCA were estimated as [(analyt's area/IS area)*IS concentration]. The presence of bile acids in blood is characteristic only for peroxisomal biogenesis disorders (PBD) therefore calculated indicative values would be sufficient for discrimination between PBD and X-ALD patients. Assay of inter/intraday variability of DHCA and THCA concentrations was carried out through measurements of endogenous concentrations in one plasma sample from a PBD patient (CQ1).

The plasma QCs were used to evaluate the effect of three freeze-thaw cycles. One DBS sample was kept for 6 months in ambient condition and at −20° C. to evaluate the influence of store conditions on the stability of analytes. Serum and plasma samples from one subject were also tested to see possible differences in the content of analytes.

### Sample Preparation

### Plasma

50 μL of plasma sample were placed in 1.5 mL collection tube containing 10 μL of internal standard solution mix. To extract all analytes and precipitate plasma proteins 500 μL of extraction solution was added. After 5 sec mixing sample was sonicated in bath for 6 min and centrifuged for 9 min at 13.000 rpm. The supernatant solution was transferred in HPLC glass vial and evaporated under nitrogen stream. 100 μL of pure methanol was added to reconstitute the sample and after 2 sec mixing, all sample was placed in HPLC glass vial with insert.

### DBS

A 3.2 mm DBS punch was placed in 96 well plate and 200 μl of internal standard solution in methanol was added. The sealed plate was incubated on thermomixer at 40° C. and 400 rpm for 1 hour. After that methanol extract was transferred in a glass vial for injection.

### UHPLC-MS/MS

Analysis of all species was performed using UHPLC-MS/MS on ExionLC™ and QTRAP 6500+system (AB Sciex LLC, Framingham, MA, USA). The revers-phase chromatography was carried on Gemini C6-Phenyl 100×5 mm column with a 3 μm particle size (Phenomenex, Torrance, CA, USA) maintained at 50° C. Mobile phase was composed of acetonitrile (phase A—40% and phase B—95%) in water, with 10 mM ammonium formate and 0.1% formic acid. The chromatographic gradient was defined as reported in Table 5 with 0.6 ml/min flow.

TABLE 5

| Time (min) | Phase A | Phase B |
|---|---|---|
| 0.00 | 90 | 10 |
| 1.00 | 90 | 10 |
| 2.00 | 80 | 20 |
| 3.30 | 15 | 85 |
| 3.31 | 10 | 90 |
| 4.00 | 10 | 90 |
| 5.00 | 10 | 90 |
| 5.01 | 90 | 10 |
| 5.40 | 90 | 10 |

Mass spectrometry detection was conducted both in positive and negative modes using an electrospray ionization source (ESI). The source temperature was set to 550° C.; Curtain Gas-20; Ion Source Gas1-45; Ion Source Gas2-45; Collision Gas-high. All parameters were optimized by the direct infusion of standard solutions into mass spectrometry to obtain a better signal. A scheduled acquisition mode was chosen to increase the sensitivity and simplify the view of chromatograms excluding isobar species. Table 6 contains a list of MRM transitions for all analytes and mass spectrometry settings used.

TABLE 6

| | | | | | |
|---|---|---|---|---|---|
| | Q1 | Q3 | DP | CE | CXP |
| ID transition | (Da) | (Da) | (V) | (V) | (V) |

Positive ionization mode 5500 V

| | | | | | |
|---|---|---|---|---|---|
| LysoGM1-1 | 1280.7 | 204.2 | 220 | 70 | 10 |
| LysoGM1-2 | 1280.7 | 366.3 | 220 | 57 | 10 |
| LysoGM1-3* | 1280.7 | 989.5 | 220 | 50 | 10 |
| LysoGB3 | 786.4 | 282.4 | 120 | 53 | 10 |
| Lyso509 | 509.5 | 184.1 | 120 | 35 | 10 |
| LysoSM | 465.4 | 184.1 | 100 | 30 | 9 |
| LysoHexSph | 462.4 | 282.3 | 60 | 30 | 14 |
| LysoSM-D7 | 472.4 | 184.2 | 100 | 30 | 9 |
| LPC26:0 | 636.5 | 184.3 | 50 | 40 | 9 |
| LPC26:0-1* | 636.5 | 104.0 | 50 | 40 | 9 |
| LysoGM2-1 | 1118.5 | 827.0 | 250 | 50 | 10 |
| LysoGM2-2* | 1118.5 | 282.3 | 250 | 60 | 10 |
| LPC26:0-D4 | 640.9 | 184.0 | 50 | 40 | 9 |

Negative ionization mode −4500 V

| | | | | | |
|---|---|---|---|---|---|
| C18-sulfatide | 806.4 | 97.0 | −100 | −150 | −5 |
| C16-sulfatide | 778.5 | 97.0 | −80 | −140 | −6 |
| C18-D3-sulfatide | 809.4 | 97.0 | −100 | −130 | −5 |
| C16:1-OH- | 792.5 | 97.0 | −100 | −120 | −11 |
| sulfatide | | | | | |
| C16-OH-sulfatide | 794.5 | 97.0 | −100 | −120 | −11 |
| DHCA | 433.6 | 433.6 | −170 | −5 | −11 |
| THCA | 449.5 | 449.5 | −160 | −5 | −11 |
| DHCA-D3 | 436.4 | 436.4 | −170 | −5 | −11 |
| THCA-D3 | 452.3 | 452.3 | −170 | −5 | −11 |

*transition used for quantification.
DP—declustering potential; CE—collision energy; CXP—collision cell exit potential.

### Statistical Analysis

Statistical differences between means were evaluated using Mann-Whitney U Test because of small group size in patient's populations. GraphPad Prism 3.03 was used for scattering plots.

### Results

### Method Validation

Calibration curves are presented in FIGS. 1 and 2. The correlation coefficients (R²) calculated from the calibration curves were all above 0.99 in plasma and DBS except C16-sulfatide with R² above 0.987 in DBS. A relevant basal amount of some analytes (especially C16-sulfatide and LPC26:0) in pooled plasma or blood requested adjustment of the calibration curves by subtracting the basal levels of analytes from each point of calibration curve: [pick area of calibration point-pick area of blank sample]. The accuracy of the method was assessed by performing recovery studies using the QCs. Recovery (in %) was calculated as mean amount divided by theoretical amount and multiplied by 100. Within-run and between-run precision were determined by preparing and analysing each QC 5 times per run over 3 consecutive days. For DBS analysis the precision studies included punches both from center and sides to evaluate chromatographic effects of filter paper on analytes. The results of accuracy and precision evaluation for plasma samples and for DMS samples are listed in Table 7 (a-c) and Table 8 (a-c), respectively.

### Table 7

### Precision and Accuracy Assay for Plasma Samples

TABLE 7a

| | QC 1 | | |
|---|---|---|---|
| | Conc | CV (%) | CV (%) |
| | (nM) | interd. | intrad. |
| lysoGM1 | nd | nd | nd |
| lysoGB3 | 0.29 | 12 | 12 |
| lysoSM | 6.9 | 6 | 2 |
| HexSph | 0.96 | 8 | 5 |
| LPC26:0 | 419 | 15 | 11 |
| lysoGM2 | nd | nd | nd |
| C18- | 18.7 | 8 | 2 |
| sulfa | | | |
| C16- | 294 | 19 | 12 |
| sulfa | | | |
| lyso509* | 1.10 | 6 | 5 |
| C16:1- | 0.80 | 6 | 4 |
| OH* | | | |
| C16- | 1.01 | 8 | 5 |
| OH* | | | |
| DHCA | 4505 | 16 | 2 |
| THCA | 962 | 17 | 1.3 |

*calculated MOM

TABLE 7b

QC 2

| | Conc | CV (%) | CV (%) | Recup. |
|---|---|---|---|---|
| | (nM) | interd. | intrad. | (%) |
| lysoGM1 | 10.0 | 4 | 1 | 100 |
| lysoGB3 | 2.3 | 5 | 4 | 101 |
| lysoSM | 16.5 | 2 | 2 | 96 |
| HexSph | 11.2 | 7 | 3 | 102 |
| LPC26:0 | 540 | 9 | 4 | 121 |
| lysoGM2 | 9.5 | 9 | 8 | 95 |
| C18- | 40.2 | 6 | 3 | 108 |
| sulfa | | | | |
| C16- | 416 | 17 | 8 | 122 |
| sulfa | | | | |
| lyso509* | 1.02 | 9 | 5 | nd |
| C16:1- | 0.86 | 6 | 5 | nd |
| OH* | | | | |
| C16- | 0.93 | 9 | 4 | nd |
| OH* | | | | |
| DHCA | — | — | — | — |
| THCA | — | — | — | — |

*calculated MOM

TABLE 7c

QC 3

| | Conc | CV (%) | CV (%) | Recup. |
|---|---|---|---|---|
| | (nM) | interd. | intrad. | (%) |
| lysoGM1 | 95.7 | 5 | 4 | 96 |
| lysoGB3 | 19.3 | 6 | 4 | 95 |
| lysoSM | 107 | 1 | 2 | 100 |
| HexSph | 105 | 7 | 4 | 104 |
| LPC26:0 | 1600 | 6 | 3 | 118 |
| lysoGM2 | 97.3 | 9 | 8 | 97 |
| C18- | 237 | 7 | 4 | 109 |
| sulfa | | | | |
| C16- | 1500 | 12 | 10 | 121 |
| sulfa | | | | |
| lyso509* | 1.10 | 11 | 3 | nd |
| C16:1- | 0.90 | 8 | 4 | nd |
| OH* | | | | |
| C16- | 0.94 | 9 | 5 | nd |
| OH* | | | | |
| DHCA | — | — | — | — |
| THCA | — | — | — | — |

*calculated MOM

### Table 8

### Precision and Accuracy Assay for DBS Samples

TABLE 8a

| | QC 1 | | |
|---|---|---|---|
| | Conc | CV (%) | CV (%) |
| | (nM) | interd. | intrad. |
| lysoGM1 | nd | nd | nd |
| lysoGB3 | nd | nd | nd |
| lysoSM | 12.5 | 23 | 16 |
| HexSph | 12.2 | 13 | 9 |
| LPC26:0 | 31.8 | 13 | 10 |
| lysoGM2 | nd | nd | nd |
| C18-sulfa | 12.6 | 16 | 8 |
| C16-sulfa | 332 | 11 | 5 |
| lyso509* | 0.46 | 10 | 4 |
| C16:1- | 2.08 | 6 | 4 |
| OH* | | | |
| C16-OH* | 1.73 | 4 | 5 |
| DHCA | nd | nd | nd |
| THCA | nd | nd | nd |

*calculated MOM

TABLE 8b

QC 2

| | Conc | CV (%) | CV (%) | Recup. |
|---|---|---|---|---|
| | (nM) | interd. | intrad. | (%) |
| lysoGM1 | 281 | 18 | 15 | 112 |
| lysoGB3 | 105 | 13 | 10 | 105 |
| lysoSM | 96.2 | 5 | 3 | 84 |
| HexSph | 270 | 7 | 4 | 103 |
| LPC26:0 | 312 | 24 | 1 | 112 |
| lysoGM2 | 219 | 27 | 28 | 88 |
| C18-sulfa | 298 | 3 | 2 | 114 |
| C16-sulfa | 549 | 8 | 4 | 87 |
| lyso509* | 0.50 | 5 | 5 | nd |
| C16:1- | 1.96 | 8 | 3 | nd |
| OH* | | | | |
| C16-OH* | 1.71 | 7 | 2 | nd |
| DHCA | — | — | — | — |
| THCA | — | — | — | — |

*calculated MOM

TABLE 8c

QC 3

| | Conc | CV (%) | CV (%) | Recup. |
|---|---|---|---|---|
| | (nM) | interd. | intrad. | (%) |
| lysoGM1 | 578 | 13 | 13 | 116 |
| lysoGB3 | 176 | 10 | 6 | 88 |
| lysoSM | 181 | 9 | 6 | 84 |
| HexSph | 462 | 12 | 2 | 90 |
| LPC26:0 | 560 | 21 | 2 | 106 |
| lysoGM2 | 426 | 18 | 24 | 85 |
| C18-sulfa | 502 | 3 | 3 | 98 |
| C16-sulfa | 825 | 10 | 4 | 99 |
| lyso509* | 0.60 | 7 | 4 | nd |
| C16:1-OH* | 1.99 | 8 | 4 | nd |
| C16-OH* | 1.61 | 5 | 3 | nd |
| DHCA | — | — | — | — |
| THCA | — | — | — | — |

*calculated MOM

The relative errors in accuracy measurements were below 22%. The CVs for all analytes in plasma did not exceed 20%, while for DBS the inter-day CVs for LysoSM, and LPC26:0 were between 20-25% in some QCs and all QCs of LysoGM2 had CVs in the range 18-28%. This variation can result from less homogeneous distribution of analytes on filter paper and poor ionization efficiency of LysoGM2 that lead to increased errors during pick integration.

The limit of detection (LOD) and quantification (LOQ) for each biomarker was determined using a signal-to-noise ratio of 3 or 10 respectively and are shown in Table 9. No significant changes were obtained in analyte concentrations after three freeze-thaw cycles in plasma samples or between DBS kept for 6 months in ambient conditions or at −20° C. In addition, no differences were obtained in the concentrations between serum and plasma samples.

TABLE 9
Limits of detection (LOD) and quantification (LOQ)

| | |
|---|---|
| Plasma | DBS |

| | LOD (nM) | LOQ (nM) | LOD (nM) | LOQ (nM) |
|---|---|---|---|---|
| lysoGM1 | 0.6 | 2.0 | 3.5 | 12 |
| lysoGB3 | 0.2 | 0.4 | 0.7 | 2.1 |
| lysoSM | 0.2 | 0.6 | 0.5 | 1.5 |
| HexSph | 0.3 | 1.0 | 2.5 | 8 |
| LPC26:0 | 0.2 | 0.7 | 1.7 | 5 |
| lysoGM2 | 2.7 | 8.25 | 60 | 125 |
| C18-sulfa | 0.5 | 1.5 | 1.0 | 3.5 |
| C16-sulfa | 10 | 30 | 10 | 31 |
| DHCA | 20 | 60 | nd | nd |
| THCA | 5 | 2 | nd | nd |

### Quantification of Biomarkers

The resulting tables demonstrating the levels of biomarkers in plasma and DBS in controls and patients are reported in Table 10 (a-c) for plasma and in Table 11 (a-c) for DBS. For plasma

TABLE 10a

| | | | LysoGM1 | | LysoGM2 | | LysoGB3 | |
|---|---|---|---|---|---|---|---|---|
| | | | (nM) | | (nM) | | (nM) | |
| | | | me- | | me- | | me- | |
| | n | Age | dian | range | dian | range | dian | range |
| Controls | 122 | 0.1- | nd | nd | nd | nd | 0.4 | 0.2- |
| | | 63.3 | | | | | | 0.7 |
| GM1 | 6 | 0.7- | 1.5 | 0.6- | nd | nd | 0.6 | 0.3- |
| gangliosidosis | | 19.8 | | 93.5 | | | | 1.2 |
| GM2 | 4 | 1.8- | nd | nd | 55.4 | 3.8- | 0.7 | 0.4- |
| gangliosidosis | | 3.1 | | | | 104 | | 1.1 |
| Tay-Sachs | 2 | 2.6- | nd | nd | 53.8 | 3.8- | 0.5 | 0.4- |
| | | 3.1 | | | | 104 | | 0.5 |
| Sandhoff | 2 | 1.8- | nd | nd | 55.4 | 49.9- | 0.9 | 0.8- |
| | | 2.1 | | | | 60.9 | | 1.1 |
| Fabry - | 2 | 2.2- | nd | nd | nd | nd | 5.5 | 4.4- |
| female | | 10.9 | | | | | | 6.5 |
| Fabry - | 2 | 9.7- | nd | nd | nd | nd | 206 | 39.5- |
| male | | 10.1 | | | | | | 373 |
| Krabbe - | 2 | 0.1- | nd | nd | nd | nd | 0.5 | 0.4- |
| classical | | 0.8 | | | | | | 0.6 |
| form | | | | | | | | |
| Krabbe - | 4 | 0.8- | nd | nd | nd | nd | 0.5 | 0.3- |
| post | | 2.8 | | | | | | 0.7 |
| transplant | | | | | | | | |
| Gaucher - | 2 | 0.1- | nd | nd | nd | nd | 3.4 | 2.3- |
| naïve | | 15.3 | | | | | | 4.4 |
| ASMD | 6 | 8.1- | nd | nd | nd | nd | 0.4 | 0.3- |
| | | 17.8 | | | | | | 0.7 |
| Niemann- | 23 | 0.3- | nd | nd | nd | nd | 0.4 | 0.2- |
| Pick - C | | 28.2 | | | | | | 0.7 |
| ALD | 8 | 4.2- | nd | nd | nd | nd | 0.5 | 0.3- |
| | | 28.9 | | | | | | 0.8 |
| AMN | 11 | 34.7- | nd | nd | nd | nd | 0.6 | 0.4- |
| | | 63.2 | | | | | | 1.0 |
| ALD carrier | 5 | 36.7- | nd | nd | nd | nd | 0.6 | 0.4- |
| | | 71.8 | | | | | | 0.9 |
| PBD | 6 | 0.1- | nd | nd | nd | nd | 0.4 | 0.2- |
| | | 20.4 | | | | | | 0.5 |
| MEDNIK, | 4 | 1.2- | nd | nd | nd | nd | 0.4 | 0.2- |
| MEDNIK- | | 15.8 | | | | | | 0.5 |
| like | | | | | | | | |
| LAL | 5 | 0.5- | nd | nd | nd | nd | 0.4 | 0.3- |
| | | 14.8 | | | | | | 0.7 |
| MLD - | 2 | 2.8- | nd | nd | nd | nd | 0.4 | 0.4- |
| late | | 3.1 | | | | | | 0.5 |
| infantile | | | | | | | | |
| MLD - | 4 | 5.8- | nd | nd | nd | nd | 0.4 | 0.3- |
| juvenile | | 13.2 | | | | | | 0.5 |

TABLE 10b

| | | | LysoHexSph | | LysoSM | | LysoSM-509 | | LPC 26:0 | |
|---|---|---|---|---|---|---|---|---|---|---|
| | | | (nM) | | (nM) | | MOM | | (nM) | |
| | n | Age | median | range | median | range | median | range | median | range |
| Controls | 122 | 0.1- | 2.4 | 0.7- | 8.7 | 3.9- | 1.0 | 0.5- | 300 | 167- |
| | | 63.3 | | 6.1 | | 18.4 | | 3.5 | | 478 |
| GM1 | 6 | 0.7- | 4.9 | 1.0- | 20.5 | 4.3- | 3.1 | 0.8- | 363 | 187- |
| gangliosidosis | | 19.8 | | 9.2 | | 31.7 | | 8.6 | | 482 |
| GM2 | 4 | 1.8- | 1.0 | 0.7- | 4.1 | 2.2- | 3.8 | 0.8- | 322 | 190- |
| gangliosidosis | | 3.1 | | 1.1 | | 6.2 | | 10.1 | | 420 |
| Tay-Sachs | 2 | 2.6- | 1.0 | 1.0- | 4.8 | 3.5- | 5.5 | 0.8- | 322 | 256- |
| | | 3.1 | | 1.1 | | 6.2 | | 10.1 | | 389 |
| Sandhoff | 2 | 1.8- | 0.8 | 0.7- | 3.4 | 2.2- | 3.8 | 1.5- | 305 | 190- |
| | | 2.1 | | 1.0 | | 4.6 | | 6.0 | | 420 |
| Fabry - | 2 | 2.2- | 1.0 | 0.9- | 4.7 | 4.2- | 1.0 | 0.9- | 174 | 147- |
| female | | 10.9 | | 1.0 | | 5.1 | | 1.1 | | 202 |
| Fabry - | 2 | 9.7- | 1.2 | 1.1- | 8.4 | 7.1- | 5.5 | 3.8- | 208 | 128- |
| male | | 10.1 | | 1.2 | | 9.8 | | 7.3 | | 289 |
| Krabbe - | 2 | 0.1- | 35.0 | 28.9- | 6.3 | 5.1- | 2.2 | 1.2- | 363 | 320- |
| classical | | 0.8 | | 41.1 | | 7.6 | | 3.3 | | 407 |
| form | | | | | | | | | | |
| Krabbe - | 4 | 0.8- | 10.9 | 9.8- | 5.0 | 3.6- | 1.2 | 0.5- | 210 | 135- |
| post | | 2.8 | | 11.7 | | 9.2 | | 1.4 | | 402 |
| transplant | | | | | | | | | | |
| Gaucher - | 2 | 0.1- | 790 | 317- | 26.6 | 6.2- | 8.3 | 4.6- | 310 | 296- |
| naïve | | 15.3 | | 1263 | | 47 | | 12.0 | | 324 |
| ASMD | 6 | 8.1- | 1.3 | 0.7- | 809 | 63- | 763 | 93- | 284 | 201- |
| | | 17.8 | | 1.8 | | 2088 | | 1281 | | 415 |
| Niemann- | 23 | 0.3- | 1.7 | 0.8- | 26.1 | 10.8- | 226 | 77- | 251 | 114- |
| Pick - C | | 28.2 | | 5.7 | | 120 | | 432 | | 391 |
| ALD | 8 | 4.2- | 1.2 | 1.1- | 9.3 | 7.9- | 1.5 | 0.9- | 2980 | 2013- |
| | | 28.9 | | 1.5 | | 16.5 | | 3.2 | | 4248 |
| AMN | 11 | 34.7- | 1.9 | 1.1- | 16.7 | 9.1- | 2.4 | 1.4- | 2788 | 1253- |
| | | 63.2 | | 2.7 | | 20.1 | | 5.5 | | 6548 |
| ALD | 5 | 36.7- | 1.4 | 1.3- | 13.2 | 9.1- | 2.3 | 1.1- | 2433 | 1463- |
| carrier | | 71.8 | | 2.5 | | 18.1 | | 2.9 | | 3433 |
| PBD | 6 | 0.1- | 1.1 | 1.0- | 7.4 | 5.8- | 2.1 | 0.8- | 8573 | 6098- |
| | | 20.4 | | 1.8 | | 13.2 | | 3.0 | | 19098 |
| MEDNIK, | 4 | 1.2- | 1.5 | 0.9- | 7.4 | 3.8- | 1.4 | 1.0- | 416 | 192- |
| MEDNIK- | | 15.8 | | 1.9 | | 8.3 | | 5.0 | | 683 |
| like | | | | | | | | | | |
| LAL | 5 | 0.5- | 3.1 | 1.8- | 13.8 | 8.0- | 21.2 | 8.4- | 503 | 258- |
| | | 14.8 | | 4.8 | | 25.1 | | 89.1 | | 580 |
| MLD - | 2 | 2.8- | 1.2 | 1.1- | 9.6 | 9.1- | 2.3 | 1.1- | 520 | 420- |
| late | | 3.1 | | 1.3 | | 10.0 | | 3.5 | | 621 |
| infantile | | | | | | | | | | |
| MLD - | 4 | 5.8- | 0.8 | 0.5- | 8.2 | 5.1- | 0.8 | 0.6- | 358 | 196- |
| juvenile | | 13.2 | | 1.1 | | 12.6 | | 1.4 | | 558 |

TABLE 10c

| | | | Sulfatide | | Sulfatide | | Sulfatide | | Sulfatide | |
|---|---|---|---|---|---|---|---|---|---|---|
| | | | C18 | | C16 | | C16:1-OH | | C16-OH | |
| | | | (nM) | | (nM) | | MOM | | MOM | |
| | n | Age | median | range | median | range | median | range | median | range |
| Controls | 122 | 0.1- | 18.7 | 9.4- | 201 | 78- | 1.0 | 0.1- | 1.0 | 0.2- |
| | | 63.3 | | 48 | | 406 | | 2.2 | | 2.1 |
| GM1 | 6 | 0.7- | 18.0 | 13.5- | 217 | 127- | 1.2 | 0.5- | 1.0 | 0.6- |
| gangliosidosis | | 19.8 | | 28.5 | | 444 | | 3.5 | | 1.6 |
| GM2 | 4 | 1.8- | 35.4 | 31.8- | 218 | 100- | 1.1 | 0.4- | 0.9 | 0.5- |
| gangliosidosis | | 3.1 | | 46 | | 250 | | 1.7 | | 1.4 |
| Tay- | 2 | 2.6- | 40 | 33.3- | 218 | 196- | 1.1 | 0.7- | 1.0 | 0.6- |
| Sachs | | 3.1 | | 46.0 | | 241 | | 1.5 | | 1.4 |
| Sandhoff | 2 | 1.8- | 34.7 | 31.8- | 175 | 100- | 1.1 | 0.4- | 0.8 | 0.5- |
| | | 2.1 | | 37.5 | | 250 | | 1.7 | | 1.2 |
| Fabry - | 2 | 2.2- | 25.6 | 17.8- | 209 | 196- | 1.0 | 0.6- | 0.9 | 0.6- |
| female | | 10.9 | | 33.4 | | 222 | | 1.5 | | 1.1 |
| Fabry - | 2 | 9.7- | 17.5 | 9.9- | 215 | 129- | 1.0 | 0.5- | 1.1 | 0.7- |
| male | | 10.1 | | 25.1 | | 302 | | 1.5 | | 1.5 |
| Krabbe - | 2 | 0.1- | 84.8 | 78.7- | 236 | 215- | 1.6 | 1.0- | 2.0 | 1.0- |
| classical | | 0.8 | | 91.0 | | 258 | | 2.2 | | 3.0 |
| form | | | | | | | | | | |
| Krabbe - | 4 | 0.8- | 86.6 | 41.5- | 445 | 145- | 2.9 | 0.6- | 2.7 | 0.6- |
| post | | 2.8 | | 102 | | 463 | | 4.9 | | 3.5 |
| transplant | | | | | | | | | | |
| Gaucher - | 2 | 0.1- | 11.9 | 11.8- | 120 | 39.4- | 0.6 | 0.3- | 0.6 | 0.2- |
| naïve | | 15.3 | | 12.0 | | 200 | | 1.0 | | 1.1 |
| ASMD | 6 | 8.1- | 18.1 | 10.8- | 172 | 124- | 0.9 | 0.5- | 0.8 | 0.6- |
| | | 17.8 | | 41.1 | | 310 | | 1.2 | | 1.4 |
| Niemann- | 23 | 0.3- | 21.1 | 8.9- | 244 | 78- | 0.9 | 0.3- | 1.1 | 0.4- |
| Pick - C | | 28.2 | | 34.6 | | 502 | | 2.1 | | 2.2 |
| ALD | 8 | 4.2- | 14.9 | 9.3- | 227 | 143- | 1.0 | 0.6- | 1.2 | 0.8- |
| | | 28.9 | | 23.9 | | 358 | | 1.1 | | 1.6 |
| AMN | 11 | 34.7- | 14.1 | 10.2- | 264 | 168- | 1.6 | 1.0- | 1.3 | 1.0- |
| | | 63.2 | | 22.1 | | 583 | | 3.0 | | 2.6 |
| ALD | 5 | 36.7- | 18.8 | 15.9- | 250 | 202- | 1.5 | 1.4- | 1.6 | 1.0- |
| carrier | | 71.8 | | 22.1 | | 380 | | 2.9 | | 2.3 |
| PBD | 6 | 0.1- | 17.0 | 10.7- | 175 | 81.3- | 0.8 | 0.3- | 0.9 | 0.4- |
| | | 20.4 | | 49.8 | | 273 | | 1.4 | | 1.4 |
| MEDNIK, | 4 | 1.2- | 33.4 | 28.5- | 1434 | 1139- | 1.4 | 0.9- | 1.1 | 0.4- |
| MEDNIK- | | 15.8 | | 73.6 | | 3537 | | 2.5 | | 2.2 |
| like | | | | | | | | | | |
| LAL | 5 | 0.5- | 30.2 | 10.6- | 202 | 58.5- | 1.8 | 0.2- | 1.2 | 0.2- |
| | | 14.8 | | 37.5 | | 331 | | 2.0 | | 2.1 |
| MLD - | 2 | 2.8- | 335 | 285- | 1060 | 993- | 19.4 | 19.1- | 7.2 | 6.8- |
| late | | 3.1 | | 385 | | 1127 | | 19.7 | | 7.5 |
| infantile | | | | | | | | | | |
| MLD - | 4 | 5.8- | 37.5 | 23.8- | 342 | 273- | 5.5 | 4.5- | 2.4 | 1.9- |
| juvenile | | 13.2 | | 68.5 | | 504 | | 9.0 | | 3.4 |

TABLE 11a
Per DBS

| | | | LysoGM1 | | LysoGM2 | | LysoGB3 | |
|---|---|---|---|---|---|---|---|---|
| | | | (nM) | | (nM) | | (nM) | |
| | | | me- | | me- | | me- | |
| | n | Age | dian | range | dian | range | dian | range |
| Controls | 188 | 0.1- | nd | — | nd | — | 0.9 | <0.7- |
| | | 63.3 | | | | | | 1.6 |
| GM1 | 5 | 0.6- | nd | nd- | nd | — | nd | <0.7 |
| gangliosidosis | | 11.2 | | 8.0 | | | | |
| GM2 | 4 | 1.8- | nd | — | nd | — | nd | <0.7- |
| gangliosidosis | | 6.0 | | | | | | 1.3 |
| Fabry - | 3 | 2.2- | nd | — | nd | — | 7.0 | 6.4- |
| female | | 13.6 | | | | | | 8.9 |
| Fabry - | 1 | 9.7 | nd | — | nd | — | 84.7 | — |
| male | | | | | | | | |
| Krabbe - | 1 | 0.5 | nd | — | nd | — | nd | — |
| classical form | | | | | | | | |
| Krabbe - post | 1 | 2.8 | nd | — | nd | — | nd | — |
| transplant | | | | | | | | |
| Gaucher - | 1 | 15.3 | nd | — | nd | — | nd | — |
| naïve | | | | | | | | |
| ASMD | 2 | 0.5- | nd | — | nd | — | nd | — |
| | | 8.1 | | | | | | |
| Niemann-Pick - | 11 | 0.3- | nd | — | nd | — | 0.75 | <0.7- |
| C | | 19.8 | | | | | | 0.77 |
| ALD | 8 | 6.9- | nd | — | nd | — | 1.1 | <0.7- |
| | | 57.2 | | | | | | 1.1 |
| PBD | 6 | 4.7- | nd | — | nd | — | 1.2 | <0.7- |
| | | 19.8 | | | | | | 1.2 |
| MEDNIK, | 4 | 1.2- | nd | — | nd | — | nd | — |
| MEDNIK-like | | 15.8 | | | | | | |
| LAL | 3 | 2.1- | nd | — | nd | — | 1.5 | <0.7- |
| | | 14.5 | | | | | | 1.5 |
| MLD - | 2 | 2.8- | nd | — | nd | — | 0.8 | <0.7- |
| late infantile | | 3.1 | | | | | | 0.8 |
| MLD - | 4 | 9.5- | nd | — | nd | — | nd | — |
| juvenile | | 13.8 | | | | | | |

TABLE 11b

| | | | LysoHexSph | | LysoSM | | LysoSM-509 | | LPC 26:0 | |
|---|---|---|---|---|---|---|---|---|---|---|
| | | | (nM) | | (nM) | | MOM | | (nM) | |
| | n | Age | median | range | median | range | median | range | median | range |
| Controls | 188 | 0.1- | 4.0 | <2.5- | 43.4 | 18.6- | 1.0 | 0.4- | 28.6 | 14.6- |
| | | 63.3 | | 13.6 | | 84.5 | | 2.8 | | 92.7 |
| GM1 | 5 | 0.6- | 2.5 | 1.0- | 47.4 | 23.6- | 2.7 | 1.8- | 35.6 | 29.8- |
| gangliosidosis | | 11.2 | | 5.3 | | 76.6 | | 8.7 | | 57.9 |
| GM2 | 4 | 1.8- | 4.1 | <2.5- | 76.4 | 43.7- | 2.1 | 1.3- | 33.5 | 32.4- |
| gangliosidosis | | 6.0 | | 10.7 | | 85.4 | | 2.7 | | 39.7 |
| Fabry - | 3 | 2.2- | 1.5 | 1.3- | 45.8 | 3.5- | 1.9 | 0.3- | 36.9 | 23.1- |
| female | | 13.6 | | 2.7 | | 51.4 | | 1.9 | | 42.6 |
| Fabry - | 1 | 9.7 | 1.2 | — | 51.0 | — | 2.1 | — | 41.4 | — |
| male | | | | | | | | | | |
| Krabbe - | 1 | 0.5 | 19.6 | — | 60.6 | — | 2.0 | — | 39.0 | — |
| classical | | | | | | | | | | |
| form | | | | | | | | | | |
| Krabbe - | 1 | 2.8 | 7.5 | — | 62.6 | — | 4.1 | — | 40.4 | — |
| post | | | | | | | | | | |
| transplant | | | | | | | | | | |
| Gaucher - | 1 | 15.3 | 160.5 | — | 95.0 | — | 1.9 | — | 45.4 | — |
| naïve | | | | | | | | | | |
| ASMD | 2 | 0.5- | 2.9 | 1.9- | 942 | 652- | 30.3 | 27.7- | 72.4 | 51.6- |
| | | 8.1 | | 4.0 | | 1232 | | 33.0 | | 93.1 |
| Niemann- | 11 | 0.3- | 1.8 | 1.2- | 54.2 | 9.6- | 6.6 | 3.7- | 31.3 | 14.3- |
| Pick - C | | 19.8 | | 8.9 | | 99.4 | | 12.7 | | 54.6 |
| ALD | 8 | 6.9- | 4.5 | 1.2- | 10.2 | 6.1- | 1.0 | 0.2- | 173 | 134- |
| | | 57.2 | | 9.7 | | 16.5 | | 1.9 | | 522 |
| PBD | 6 | 4.7- | 2.7 | 1.3- | 28.8 | 6.9- | 1.5 | 0.4- | 480 | 296- |
| | | 19.8 | | 7.9 | | 42.6 | | 7.2 | | 788 |
| MEDNIK, | 4 | 1.2- | 2.8 | 0.8- | 38.8 | 4.3- | 1.4 | 0.1- | 34.6 | 17.2- |
| MEDNIK- | | 15.8 | | 4.3 | | 110 | | 3.2 | | 43.0 |
| like | | | | | | | | | | |
| LAL | 3 | 2.1- | 2.5 | 1.4- | 48.6 | 35.8- | 1.7 | 1.3- | 33.2 | 16.4- |
| | | 14.5 | | 2.5 | | 61.0 | | 6.6 | | 36.4 |
| MLD - | 2 | 2.8- | 1.3 | 1.1- | 65.2 | 61.4- | 2.4 | 2.3- | 33.5 | 32.8- |
| late | | 3.1 | | 1.5 | | 69.0 | | 2.5 | | 34.3 |
| infantile | | | | | | | | | | |
| MLD - | 4 | 9.5- | 1.5 | 0.6- | 48.8 | 27.2- | 1.9 | 1.2- | 24.9 | 22.1- |
| juvenile | | 13.8 | | 1.6 | | 69.4 | | 6.3 | | 39.2 |

TABLE 11c

| | | | Sulfatide | | Sulfatide | | Sulfatide | | Sulfatide | |
|---|---|---|---|---|---|---|---|---|---|---|
| | | | C18 | | C16 | | C16:1-OH | | C16-OH | |
| | | | (nM) | | (nM) | | MOM | | MOM | |
| | n | Age | median | range | median | range | median | range | median | range |
| Controls | 188 | 0.1- | 14.3 | 7.0- | 198 | 83.5- | 1.2 | 0.4- | 1.0 | 0.3- |
| | | 63.3 | | 29.4 | | 482 | | 2.2 | | 2.0 |
| GM1 | 5 | 0.6- | 12.7 | 10.2- | 181 | 99- | 1.5 | 0.3- | 1.1 | 0.4- |
| gangliosidosis | | 11.2 | | 15.4 | | 293 | | 4.6 | | 2.1 |
| GM2 | 4 | 1.8- | 19.8 | 15.6- | 120 | 107- | 0.9 | 0.6- | 1.0 | 0.5- |
| gangliosidosis | | 6.0 | | 24.8 | | 166 | | 1.5 | | 1.2 |
| Fabry - | 3 | 2.2- | 13.7 | 13.7- | 175 | 148- | 2.1 | 1.5- | 1.7 | 1.4- |
| female | | 13.6 | | 21.0 | | 263 | | 2.4 | | 2.5 |
| Fabry - | 1 | 9.7 | 11.9 | — | 149 | — | 1.5 | — | 1.4 | — |
| male | | | | | | | | | | |
| Krabbe - | 1 | 0.5 | 45.4 | — | 131 | — | 1.6 | — | 1.0 | — |
| classical | | | | | | | | | | |
| form | | | | | | | | | | |
| Krabbe - | 1 | 2.8 | 35.6 | — | 260 | — | 2.8 | — | 1.8 | — |
| post | | | | | | | | | | |
| transplant | | | | | | | | | | |
| Gaucher - | 1 | 15.3 | 10.8 | — | 205 | — | 1.9 | — | 1.9 | — |
| naïve | | | | | | | | | | |
| ASMD | 2 | 0.5- | 12.9 | 9.6- | 99.1 | 98.1- | 0.8 | 0.4- | 0.5 | 0.3- |
| | | 8.1 | | 16.1 | | 100 | | 1.2 | | 0.8 |
| Niemann - | 11 | 0.3- | 12.3 | 6.2- | 155 | 105- | 1.0 | 0.5- | 1.6 | 0.7- |
| Pick - C | | 19.8 | | 14.6 | | 218 | | 1.6 | | 2.0 |
| ALD | 8 | 6.9- | 8.3 | 4.5- | 122 | 82.4- | 1.0 | 0.6- | 0.8 | 0.5- |
| | | 57.2 | | 15.8 | | 208 | | 1.6 | | 1.3 |
| PBD | 6 | 4.7- | 14.2 | 8.0- | 201 | 112- | 2.0 | 1.2- | 1.8 | 1.4- |
| | | 19.8 | | 17.8 | | 256 | | 2.6 | | 3.3 |
| MEDNIK, | 4 | 1.2- | 16.9 | 13.7- | 865 | 672- | 1.3 | 0.8- | 0.8 | 0.6- |
| MEDNIK- | | 15.8 | | 32.1 | | 1562 | | 2.7 | | 2.4 |
| like | | | | | | | | | | |
| LAL | 3 | 2.1- | 13.2 | 11.6- | 157 | 110- | 1.9 | 1.5- | 1.7 | 1.2- |
| | | 14.5 | | 13.8 | | 168 | | 2.0 | | 1.9 |
| MLD - | 2 | 2.8- | 165 | 143- | 648 | 623- | 24.4 | 20.4- | 9.9 | 9.5- |
| late | | 3.1 | | 187 | | 673 | | 28.3 | | 10.2 |
| infantile | | | | | | | | | | |
| MLD - | 4 | 9.5- | 16.6 | 10.8- | 350 | 237- | 8.5 | 4.7- | 4.2 | 2.3- |
| juvenile | | 13.8 | | 28.8 | | 535 | | 10.8 | | 5.1 |

For some analytes presented in very small levels (like LysoGB3) values above LOD (but below LOQ) were included for indicative evaluation. LysoGB3 and LysoHexSph were detectable in all plasma but below LOD in some DBS. Significant increase of LysoGB3 was seen in plasma and DBS of Fabry disease with net difference between females and males (median female 5.5 nM vs 206 male nM in plasma; 7.0 female nM vs 84.7 nM male in DBS). A slight increase of LysoGB3 in plasma of Gaucher patients was also observed (median 3.4 nM vs 0.4 nM in controls), while the LysoGB3 in DBS of the 1. Gaucher patient tested was 10 below LOD.

LysoHexSph was elevated in plasma and DBS from Krabbe and Gaucher patients (35.0 nM and 790 nM in plasma; 19.6 nM and 160.5 nM in DBS respectively). Constantly high levels of C18-sulfatide were observed in plasma and DBS of Krabbe patients respect to controls (median 84.8 nM vs 18.7 nM in plasma; 45.4 nM vs 14.3 nM in DBS) while C18-sulfatide in Gaucher patients was not statistically different from controls.

Elevated LysoSM and Lyso509 levels were obtained in all plasma and DBS from ASMD and NPC patients. Plasma Lyso509 was also significantly higher in all LAL (median 21.2 vs 1.0) and Gaucher patients (8.3 vs 1.0). The levels of LysoSM and Lyso509 in plasma were relatively elevated in order: ASMD>NPC>LAL (809 nM>26.1 nM>13.8 nM for LysoSM; 763 nM>226 nM>21.2 nM for Lyso509). The same order ASMD>NPC>LAL was seen in DBS samples (942 nM>54.2 nM>48.6 nM for LysoSM; 30.3 nM>6.6 nM>1.7 nM for Lyso509). In DBS ASMD patients had the highest levels of LysoSM (942 nM vs 43.4 nM in controls) and Lyso509 (30.3 vs 1.0 in controls) while NPC patients had slightly elevated levels of Lyso509 (6.6 vs 1.0) and normal levels of LysoSM (54.2 nM vs 43.4 nM). The elevated levels of Lyso509 were seen also in single patients with GM1-gangliosidosis and peroxisomal disorders.

LPC26:0 was the only biomarker with highly different levels seen in plasma and DBS (medians in controls: 300 nM in plasma vs 28.6 nM in DBS). LPC26:0 was significantly elevated in all ALD (median 2980 nM), AMN (median 2788 nM), ALD carrier (median 2433 nM) and PBD (median 8573 nM). PBD patients had statistically higher levels of LPC26:0 respect to ALD patients (p<0.01). The bile acids were present only in plasma of PBD patients: THCA median 24 nM (5.5-9450 nM); DHCA median 329 nM (61-5450 nM). Patients with other diseases and controls had undetectable levels of THCA and DHCA. The similar results were obtained in DBS samples. However generally higher levels of LPC26:0 in DBS from PBD (median 480 nM) respect to ALD (median 173 nM) were not statistically different between total PBD and ALD populations (p<0.01). The levels of bile acids were undetectable in DBS samples.

In late-infantile MLD elevated levels of all analysed sulfatides were found in plasma: C18-sulfatide (335 nM vs 18.7 nM), C16-sulfatide (1060 nM vs 201 nM), C16:1-OH-sulfatide (19.4 vs 1.0), C16-OH-silfatide (7.2 vs 1.0). The increased levels of all sulfatides were seen also in DBS from late infantile MLD with the highest elevation for C16:1-OH-sulfatide (24.4 vs 1.2) and C16-OH-silfatide (9.9 vs 1.0). The early juvenile MLD had very moderate or absent elevation of C18-sulfatide (37.5 nM vs 18.7 nM in plasma; 16.6 nM vs 14.3 nM in DBS) and C16-sulfatide (342 nM vs 201 nM in plasma; 350 vs 198 nM in DBS) and more significant increase of C16:1-OH-sulfatide (5.5 vs 1.0 in plasma; 8.5 vs 1.2 in DBS) and C16-OH-sulfatide (2.4 vs 1.0 in plasma; 4.2 vs 1.0 in DBS).

Plasma and DBS from patients with MEDNIK syndrome-a rare disorder caused by defects in copper metabolism-were also analysed. Very high levels of C16-sulfatide were obtained in plasma (1434 nM vs 201 nM) and DBS (865 nM vs 198 nM) from MEDNIK patients while other sulfatides were in normal ranges (except C18-sulfatide [73.6 nM] elevated in one MEDNIK patient with the highest C16-sulfatide level [3537 nM]).

LysoGM1 and LysoGM2 were undetectable in the plasma and DBS of control population but well detectable in all tested plasma samples from patients with GM1-, GM2-gangliosidosis. While for DBS LysoGM1 was detectable only in 1 of 5 GM1-gangliosidosis and LysoGM2 was undetectable in all DBS from GM2-gangliosidosis because of very high LOD in DBS matrix.

### BIBLIOGRAPHY

1. Parenti G, Medina D L, Ballabio A. The rapidly evolving view of lysosomal storage diseases EMBO Mol Med. 2021 Feb. 5; 13(2): e12836.
2. Massaro M, Geard A F, Liu W, Coombe-Tennant O, Waddington S N, Baruteau J, Gissen P, Rahim A A. Gene Therapy for Lysosomal Storage Disorders: Ongoing Studies and Clinical Development Biomolecules. 2021 April; 11(4): 611.
3. Ferreira C R, Rahman S, Keller M, Zschocke J; ICIMD Advisory Group. An international classification of inherited metabolic disorders (ICIMD). J Inherit Metab Dis. 2021 January; 44 (1): 164-177. doi: 10.1002/jimd.12348.
4. Menkovic I, Boutin M, Alayoubi A, Curado F, Bauer P, Mercier F E, Rivard GÉ, Auray-Blais C. Quantitation of a plasma biomarker profile for the early detection of Gaucher disease type 1 patients. Bioanalysis. 2022 February; 14 (4): 223-240. doi: 10.4155/bio-2021-0242. Epub 2022 Feb. 4.
5. Malvagia S, Ferri L, Della Bona M, Borsini W, Cirami C L, Dervishi E, Feriozzi S, Gasperini S, Motta S, Mignani R, Trezzi B, Pieruzzi F, Morrone A, Daniotti M, Donati M A, la Marca G. Multicenter evaluation of use of dried blood spot compared to conventional plasma in measurements of globotriaosylsphingosine (LysoGb3) concentration in 104 Fabry patients. Clin Chem Lab Med. 2021 Apr. 30; 59(9): 1516-1526. doi: 10.1515/cclm-2021-0316. Print 2021 Aug. 26.
6. Hong X, Daiker J, Sadilek M, Ruiz-Schultz N, Kumar A B, Norcross S, Dansithong W, Suhr T, Escolar M L, Ronald Scott C, Rohrwasser A, Gelb M H. Toward newborn screening of metachromatic leukodystrophy: results from analysis of over 27,000 newborn dried blood spots. Genet Med. 2021 March; 23(3):555-561.
7. Su P, Khaledi H, Waggoner C, Gelb M H. Detection of GM1-gangliosidosis in newborn dried blood spots by enzyme activity and biomarker assays using tandem mass spectrometry. J Inherit Metab Dis. 2021 January; 44(1):264-271.
8. Hong X, Sadilek M, Gelb M H. A highly multiplexed biochemical assay for analytes in dried blood spots: application to newborn screening and diagnosis of lysosomal storage disorders and other inborn errors of metabolism. Genet Med. 2020 July; 22(7): 1262-1268. doi: 10.1038/s41436-020-0790-9. Epub 2020 Apr. 20.
9. Polo G, Burlina A P, Ranieri E, Colucci F, Rubert L, Pascarella A, Duro G, Tummolo A, Padoan A, Plebani M, Burlina A B. Plasma and dried blood spot lysosphingolipids for the diagnosis of different sphingolipidoses: a comparative study. Clin Chem Lab Med. 2019 Nov. 26; 57(12):1863-1874. doi: 10.1515/cclm-2018-1301.10. Natarajan A, Christopher R, Netravathi M, Bhat M, Chandra S R. Liquid chromatography-tandem mass spectrometry method for estimation of a panel of lysophosphatidylcholines in dried blood spots for screening of X-linked adrenoleukodystrophy. Clin Chim Acta. 2018 October; 485:305-310. doi: 10.1016/j.cca.2018.07.007. Epub 2018 Jul. 7.
11. Pettazzoni M, Froissart R, Pagan C, Vanier M T, Ruet S, Latour P, Guffon N, Fouilhoux A, Germain D P, Levade T, Vianey-Saban C, Piraud M, Cheillan D. LC-MS/MS multiplex analysis of lysosphingolipids in plasma and amniotic fluid: A novel tool for the screening of sphingolipidoses and Niemann-Pick type C disease. PLOS One. 2017 Jul. 27; 12(7):e0181700. doi: 10.1371/journal.pone.0181700. eCollection 2017.
12. Saville J T, Smith N J, Fletcher J M, Fuller M. Quantification of plasma sulfatides by mass spectrometry: Utility for metachromatic leukodystrophy. Anal Chim Acta. 2017 Feb. 22; 955:79-85. doi: 10.1016/j.aca.2016.12.002. Epub 2016 Dec. 19.
13. Polo G, Burlina A P, Kolamunnage T B, Zampieri M, Dionisi-Vici C, Strisciuglio P, Zaninotto M, Plebani M, Burlina A B. Diagnosis of sphingolipidoses: a new simultaneous measurement of lysosphingolipids by LC-M S/M S. Clin Chem Lab Med. 2017 Mar. 1; 55(3):403-414. doi: 10.1515/cclm-2016-0340.
14. Spacil Z, Babu Kumar A, Liao H C, Auray-Blais C, Stark S, Suhr T R, Scott C R, Turecek F, Gelb M H. Sulfatide Analysis by Mass Spectrometry for Screening of Metachromatic Leukodystrophy in Dried Blood and Urine Samples. Clin Chem. 2016 January; 62(1):279-86. doi: 10.1373/clinchem.2015.245159. Epub 2015 Nov. 19
15. Mirzaian M, Kramer G, Poorthuis B J. Quantification of sulfatides and lysosulfatides in tissues and body fluids by liquid chromatography-tandem mass spectrometry. J Lipid Res. 2015 April; 56(4):936-43. doi: 10.1194/jlr.M057232. Epub 2015 Jan. 27.
16. Moyano A L, Pituch K, Li G, van Breemen R, Mansson J E, Givogri M I. Levels of plasma sulfatides C18:0 and C24:1 correlate with disease status in relapsing-remitting multiple sclerosis. J Neurochem. 2013 December; 127(5):600-4. doi: 10.1111/jnc.12341. Epub 2013 Jul. 9.

1. A method for the in vitro diagnosis of one or more of neurodegenerative metabolic diseases selected from the group consisting of
Fabry disease, Gaucher disease, Krabbe disease, Acid sphingomyelinase deficiency, Niemann-Pick disease type C; GM1 gangliosisodis; GM2 gangliosidosis; lysosomal acid lipase deficiency; metachromatic leukodystrophy; X-linked adrenoleukodystrophy and its phenotypic variant Adrenomyeloneuropathy; MEDNIK disease; MEDNIK-like disease; peroxisomal biogenesis disorders;
said method comprising:
a) contacting a biological sample of a subject with an extracting solution able to extract all the following biomarkers: lysoGM1, lysoGM2, lysoGB3, lysoSM, lysoSM-509, lysoHexSph, LPC26:0, Sulfatide C18:0, Sulfatide C16:0, Sulfatide C16:1-OH, Sulfatide C16:0-OH, DHCA, THCA, said biological sample being plasma, dried blood spot and/or amniotic fluid in order to obtain an extracted solution;
b) detecting the presence of one or more of said biomarkers in said extracted solution by means of liquid chromatography, combined to mass spectrometry;
wherein, when said biological sample is plasma or amniotic fluid, said extracting solution comprises:
C1-C3 alcohol, such as methanol, in a percentage from 55% to 65%;
C3-C6 ketone, such as acetone, in a percentage from 25% to 35%;
water, in a percentage from 5% to 15%; whereas
when said biological sample is dried blood spot, said extracting solution comprises C1-C3 alcohol, such as methanol, in a percentage from 90% to 100%;
wherein said liquid chromatography is carried out by using
a C6-C18 reverse phase column with phenyl groups bound to the silica surface; and
a mobile phase comprising an organic solvent, such as acetonitrile, methanol, ethanol or 2-propanol and an aqueous solvent,
wherein said mass spectrometry is carried out both in positive and negative ionization modes, wherein LysoGM1, LysoGB3, Lyso509, LysoSM, LysoHexSph, LPC26:0, LysoGM2 and LPC26:0 are detected in positive ionization mode, C18-sulfatide, C16-sulfatide, C16:1-OH-sulfatide and C16-OH-sulfatide are detected in negative ionization mode and DHCA and THCA are detected in positive ionization mode or in negative ionization mode, preferably in negative ionization mode;
and wherein
lysoGM1 is for the diagnosis of GM1 gangliosisodis;
lysoGM2 is for the diagnosis of GM2 gangliosidosis;
lysoGB3 is for the diagnosis of Fabry disease;
lysoSM and lysoSM-509 are for the diagnosis of Acid sphingomyelinase deficiency and Niemann-Pick disease type C;
lysoSM-509 is for the indication of possible lysosomal acid lipase deficiency;
HexSph is for the diagnosis of Gaucher and Krabbe diseases;
LPC26:0 is for the diagnosis of X-linked adrenoleukodystrophy and peroxisomal biogenesis disorders
Sulfatide C18:0 is for the diagnosis of Metachromatic leukodystrophy and Krabbe disease;
Sulfatide C16:0 is for the diagnosis of Metachromatic leukodystrophy, MEDNIK and MEDNIK-like syndrome;
Sulfatide C16:1-OH is for the diagnosis of Metachromatic leukodystrophy;
Sulfatide C16:0-OH is for the diagnosis of Metachromatic leukodystrophy;
DHCA is for the diagnosis of peroxisomal biogenesis disorders;
THCA is for the diagnosis of peroxisomal biogenesis disorders.

2. The method according to claim 1, wherein said mobile phase further comprises a buffer solution comprising
an ammonium salt in an amount ranging from 5 to 20 mM;
a weak organic acid in an amount ranging from 0.1 to 5%; and
an aqueous solvent.

3. The method according to claim 1, wherein said mobile phase comprises:
a first phase comprising an organic solvent at a percentage of 40%, in aqueous solvent;
a second phase comprising said organic solvent at a percentage of 95%, in said aqueous solvent;

4. The method according to claim 3, wherein said first phase and said second phase further comprise a buffer solution comprising
an ammonium salt in an amount ranging from 5 to 20 mM;
a weak organic acid in an amount ranging from 0.1 to 5%; and
an aqueous solvent.

5. The method according to claim 1, wherein said liquid chromatography combined with mass spectrometry are carried out both in positive and negative modes is carried out using an electrospray ionization source and in scheduled acquisition mode.

6. The method according to claim 1, wherein said method is carried out on pre-natal, neo-natal or post-natal biological sample.

7. A kit for the in vitro diagnosis of one or more neurodegenerative diseases selected from the group consisting of
Fabry disease, Gaucher disease, Krabbe disease, Acid sphingomyelinase deficiency, Niemann-Pick disease type C; GM1 gangliosisodis; GM2 gangliosidosis; lysosomal acid lipase deficiency; metachromatic leukodystrophy; X-linked adrenoleukodystrophy and its phenotypic variant Adrenomyeloneuropathy; MEDNIK disease; MEDNIK-like disease; and peroxisomal biogenesis disorders;
said kit comprising:
i) an extraction solution comprising:
C1-C3 alcohol in a percentage from 55% to 65%;
C3-C6 ketone in a percentage from 25% to 35%;
water, in a percentage from 5% to 15%;
and/or an extracting solution comprising a C1-C3 alcohol in a percentage from 90% to 100%;
ii) a mobile phase comprising an organic solvent and an aqueous solvent or suitable ingredients able to provide said mobile phase when mixed with an aqueous solvent; and
iii) a C6-C18 reverse phase column.

8. A kit according to claim 7, wherein said kit further comprises one or more pooled samples added with standard concentrations of biomarkers.

9. A kit according to claim 7, wherein said kit further comprises a buffer solution comprising
an ammonium salt in an amount ranging from 5 to 20 mM;
a weak organic acid in an amount ranging from 0.1 to 5%; and
an aqueous solvent.

10. A kit according to claim 7, wherein the mobile phase comprises;
a first phase comprising an organic solvent at a percentage of 40%, in aqueous solvent;
a second phase comprising said organic solvent at a percentage of 95% in said aqueous solvent.

11. A method for the in vitro diagnosis of Krabbe disease, said method comprising detecting in a biological sample of a subject sulfatide C18 and LysoHexSph,
wherein a higher concentration of sulfatide C18 and LysoHexSph in said biological sample with respect to the concentration of sulfatide C18 and LysoHexSph in a biological sample of a healthy subject indicates Krabbe disease, said biological sample being plasma, dried blood and/or amniotic fluid.

12.-13. (canceled)

14. A method for the in vitro diagnosis of MEDNIK or MEDNIK-like diseases, said method comprising detecting in a biological sample of a patient sulfatide C16,
wherein a higher concentration of sulfatide C16 in said biological sample with respect to the concentration of sulfatide C16 in a biological sample of a healthy subject indicates MEDNIK or MEDNIK-like diseases, said biological sample being plasma, dried blood and/or amniotic fluid.

15. (canceled)

16. The method according to claim 1, wherein said biological sample is plasma, said liquid chromatography is ultra-high performance liquid chromatography (UPLC), said mass spectrometry is tandem mass spectrometry (MS/MS);
wherein the extracting solution comprises 60% methanol and 30% acetone;
and wherein the C6-C18 reverse phase column is a C6-C8 reverse phase column with phenyl groups bound to the silica surface; and said mobile phase comprises acetonitrile, methanol, ethanol or 2-propanol, and the aqueous solvent is water.

17. The method according to claim 2, wherein said ammonium salt is ammonium formate or ammonium acetate; the weak organic acid is formic acid, acetic acid, trifluoroacetic or perfluoroeptanoic acid; and the aqueous solvent is water.

18. The method according to claim 3, wherein said mobile phase comprises a first phase comprising acetonitrile, methanol, ethanol or 2-propanol at a percentage of 40%, in water; and
a second phase comprising acetonitrile, methanol, ethanol or 2-propanol at a percentage of 95%, in water.

19. The method according to claim 4, wherein said buffer solution comprises ammonium formate or ammonium acetate in an amount ranging from 5 to 20 mM;
formic acid, acetic acid, trifluoroacetic or perfluoroeptanoic acid in an amount ranging from 0.1 to 5%; and
water.

20. The method according to claim 5, wherein the electrospray ionization source is settled at 550° C.

21. The kit according to claim 7, wherein
i) the extraction solution comprises 60% methanol, 30% acetone, 10% water;
ii) the mobile phase comprises acetonitrile, methanol, ethanol or 2-propanol and an aqueous solvent or suitable ingredients able to provide said mobile phase when mixed with an aqueous solvent; and
iii) a C6-C8 reverse phase column with phenyl groups bound to the silica surface.

22. The kit according to claim 8, wherein the one or more pooled samples are added with a standard mixture of biomarkers for calibration, or one or more pooled samples are added with two different concentrations of a biomarker as control.

23. The kit according to claim 9, wherein said buffer solution comprises
ammonium formate or ammonium acetate in an amount ranging from 5 to 20 mM;
formic acid, acetic acid, trifluoroacetic or perfluoroeptanoic acid in an amount ranging from 0.1 to 5%; and
water.

24. The kit according to claim 10, wherein said mobile phase comprises a first phase comprising acetonitrile, methanol, ethanol or 2-propanol at a percentage of 40%, in water; and
a second phase comprising acetonitrile, methanol, ethanol or 2-propanol at a percentage of 95%, in water.
